(19) United States
(12) Patent Application Publication
(10) Pub. No.: US 2009/0149511 A1
(43) Pub. Date: Jun. 11, 2009

(54) Administration of an Inhibitor of HDAC and an mTOR Inhibitor

(75) Inventors: Konrad Burk, Madison, CT (US); Peter Ordentlich, San Diego, CA (US); Robert Goodenow, San Clemente, CA (US)

Correspondence Address: WILSON SONSINI GOODRICH & ROSATI, 650 PAGE MILL ROAD, PALO ALTO, CA, 94304-1050, US

(73) Assignee: SYNDAX PHARMACEUTICALS, INC., San Diego, CA (US)

(21) Appl. No.: 12/260,883

(22) Filed: Oct. 29, 2008

Related U.S. Application Data
(60) Provisional application No. 60/983,892, filed on Oct. 30, 2007.

(51) Int. Cl. A61K 31/44; A61P 35/04

(52) U.S. Cl. 514/357

(57) ABSTRACT

Methods of treating patients with an HDAC inhibitor and an mTOR inhibitor are provided.

### CROSS-REFERENCE

This application claims the benefit of U.S. Provisional Application No. 60/983,892, fled Oct. 30, 2007, which is incorporated herein by reference in its entirety.

### SUMMARY OF THE INVENTION

The inventors have identified a need for methods of administering an HDAC inhibitor and an mTOR inhibitor. The present invention meets this need and provides related advantages as well.

In some embodiments, the invention relates to a method of treating cancer in a patient, comprising administering an HDAC inhibitor and an mTOR inhibitor.

In some embodiments, the HDAC inhibitor is a Class I HDAC inhibitor. In some embodiments, the HDAC inhibitor is SNDX-275. In various embodiments, the SNDX-275 provides a mean area under the blood plasma concentration curve of SNDX-275 of about 25 to about 700 ng·h/mL. In some embodiments, the SNDX-275 provides a mean area under the plasma concentration curve of SNDX-275 of about 100 ng·h/mL to about 400 ng·h/mL. In some embodiments, the SNDX-275 provides a mean area under the plasma concentration curve of SNDX-275 of about 150 ng·h/mL to about 350 ng·h/mL. In some embodiments, the SNDX-275 provides a mean area under the plasma concentration curve of SNDX-275 of about 75 to about 225 ng·h/mL. In various embodiments, the mean maximum plasma concentration of SNDX-275 is between about 1 and about 50 ng/mL. In some embodiments, the mean maximum plasma concentration of SNDX-275 is between about 5 and about 25 ng/mL. In various embodiments, the mean ½ life of the SNDX-275 is greater than about 24 hours.

In some embodiments, the method further comprises detecting a drug-related toxicity in the patient and subsequently administering to the patient a reduced dose of SNDX-275.

In some embodiments, the dose of SNDX-275 is about 1 mg to about 6 mg. In some embodiments, the SNDX is administered once a week. In some embodiments, the SNDX is administered once every two weeks. In some embodiments, the mean time to maximum plasma concentration of SNDX-275 is about 0.5 to about 24 hours. In some embodiments, the SNDX-275 is administered orally in the form of one or more tablets. In some embodiments, the SNDX-275 is administered orally in the form of 0.5, 1, 2, 3, 4, 5, 6, 7, 8, 9 or 10 mg tablets or a suitable combination of two or more thereof.

In various embodiments, the mTOR inhibitor is selected from temsirolimus (CCI-779), everolimus (RAD001), deforolimus (AP23573), AP21967, biolimus, AP23102, zotarolimus (ABT 578), sirolimus (Rapamune), and tacrolimus (Prograf). The mTOR inhibitor is administered in an amount of about 0.5 to about 10 mg/day. In some embodiments, the mTOR inhibitor is administered in an amount of about 0.9 to about 4 mg/day.

In some embodiments, the cancer is of epithelial origin. In other embodiments, the cancer is a hematological cancer. In various embodiments, the cancer is lung cancer, multiple myeloma, gynecologic malignancies, non-hodgkins lymphoma, Hodgkin's disease, leukemia, melanoma, breast cancer, prostate cancer, kidney cancer, head cancer, neck cancer, renal cell cancer, or a solid tumor.

In some embodiments, provided herein are methods of treating cancer in a patient, comprising: (a) administering to the patient a first dose of 3-10 mgs of SNDX-275 and a second dose of 3-10 mgs of SNDX-275, wherein the second dose of SNDX-275 is administered within 1-3 weeks of the first dose of SNDX-275; and (b) administering at least one dose of an mTOR inhibitor, wherein the mTOR inhibitor is administered within the three weeks of the first dose of SNDX-275.

In some embodiments, the first dose of SNDX-275 provides a mean area under the blood plasma concentration curve of SNDX-275 of about 25 to about 700 ng·h/mL. In some embodiments, the first dose of SNDX-275 provides a mean area under the plasma concentration curve of SNDX-275 of about 100 ng·h/mL to about 400 ng·h/mL. In some embodiments, the first dose of SNDX-275 provides a mean area under the plasma concentration curve of SNDX-275 of about 150 ng·h/mL to about 350 ng·h/mL. In some embodiments, the first dose of SNDX-275 provides a mean area under the plasma concentration curve of SNDX-275 of about 75 to about 225 ng·h/mL. In some embodiments, the mean maximum plasma concentration of SNDX-275 is between about 1 and about 50 ng/mL. In some embodiments, the mean maximum plasma concentration of SNDX-275 is between about 5 and about 25 ng/mL. In some embodiment, the mean ½ life of the SNDX-275 is greater than about 24 hours.

In some embodiments, the method further comprises detecting a drug-related toxicity in the patient and subsequently administering to the patient a reduced dose of SNDX-275.

In some embodiments, the SNDX is administered once a week. In some embodiments, the SNDX is administered once every two weeks.

In some embodiments, the mean time to maximum plasma concentration of SNDX-275 is about 0.5 to about 24 hours.

In some embodiments, the SNDX-275 is administered orally in the form of one or more tablets.

In various embodiments, the mTOR inhibitor is selected from temsirolimus (CCI-779), everolimus (RAD001), deforolimus (AP23573), AP21967, biolimus, AP23102, zotarolimus (ABT 578), sirolimus (Rapamune), and tacrolimus (Prograf). In some embodiments, the mTOR inhibitor is sirolimus. In some embodiments, the sirolimus is administered in an amount of about 0.5 to about 10 mg/day. In some embodiments, the sirolimus is administered in an amount of about 0.9 to about 4 mg/day.

In some embodiments, the cancer is of epithelial origin. In other embodiments, the cancer is a hematological cancer. In various embodiments, the cancer is lung cancer, multiple myeloma, gynecologic malignancies, non-hodgkins lymphoma, Hodgkin's disease, leukemia, melanoma, breast cancer, prostate cancer, kidney cancer, head cancer, neck cancer, renal cell cancer, or a solid tumor.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The mammalian target of rapamycin, commonly known as mTOR, is a serine/threonine protein kinase that regulates cell growth, cell proliferation, cell motility, cell survival, protein synthesis, and transcription. mTor inhibitors, including rapamycin and its analogues, are a class of therapeutics that specifically inhibit signaling from mTOR. mTOR is a key intermediary in multiple mitogenic signaling pathways and plays a central role in modulating proliferation and angiogenesis in normal tissues and neoplastic processes. Hyperactivation of mTOR signaling has been implicated in tumorigenesis, and studies in several tumor types suggest that the anti-proliferative and anti-angiogenic properties of mTOR inhibitors are useful in cancer therapy.

DNA in eukaryotic cells is tightly complexed with proteins to form chromatin. Histones are small proteins that are tightly complexed with DNA to form a nucleosome, which is further connected by linker DNA to form a solenoid. Histones extending from the nucleosomal core are enzymatically modified, affecting chromatin structure and gene expression. The study of inhibitors of histone deacetylases (HDACs) indicates that these enzymes play an important role in cell proliferation and differentiation. The apparent involvement of HDACs in the control of cell proliferation and differentiation suggests that aberrant HDAC activity may play a role in cancer.

Histone hyperacetylation by HDAC inhibition neutralizes the positive charge of the lysine side chain, and is associated with change of the chromatin structure and the consequential transcriptional activation of a number of genes. It is believed that one outcome of histone hyperacetylation is induction of the Cyclin-dependent kinase inhibitory protein, P21, which causes cell cycle arrest. HDAC inhibitors such as Trichostatin A (TSA) and suberoylanilide hydroxamic acid (SAHA) have been reported to inhibit cell growth, induce terminal differentiation in tumor cells and prevent the formation of tumors in mice. HDACs have been viewed as attractive targets for anticancer drug development with their ability to block angiogenesis and cell cycling, and promote apoptosis and differentiation.

Compounds and compositions capable of inhibiting histone deacetylating enzymes and inducing differentiation are useful as therapeutic or ameliorating agents for diseases that are involved in cellular growth such as malignant tumors, autoimmune diseases, skin diseases, infections, other anti-proliferative therapies, etc. HDAC inhibitors are able to target the transcription of specific disease-causing genes as well as improve the efficacy of existing cytostatics (such as the retinoids). Due to its role in the transcriptional mechanism to affect the gene expression, HDAC inhibitors are also useful as a therapeutic or prophylactic agent for diseases caused by abnormal gene expression such as inflammatory disorders, diabetes, diabetic complications, homozygous thalassemia, fibrosis, cirrhosis, acute promyelocytic leukemia (APL), organ transplant rejections, autoimmune diseases, protozoal infections, tumors, etc.

Provided herein is a method of treating a disease state, in particular cancer, by administering to a patient in need of such treatment an effective dose of an HDAC inhibitor and an mTOR inhibitor. In some embodiments, the HDAC inhibitor is a Class I Selective HDAC inhibitor. In some embodiments, the HDAC inhibitor is SNDX-275. In some embodiments, the cancer is a solid tumor; in others it is a hematological malignancy (e.g., leukemia). In particular embodiments, the mode of administration is oral administration for at least one of the HDAC inhibitor and the mTOR inhibitor. In some embodiments, the mode of administration is oral for both the HDAC inhibitor and the mTOR inhibitor.

### Certain Terminology

Unless defined otherwise, all technical and scientific terms used herein have the same meaning as is commonly understood by one of skill in the art to which the claimed subject matter belongs. In the event that there is a plurality of definitions for terms herein, those in this section prevail. Where reference is made to a URL or other such identifier or address, it is understood that such identifiers can change and particular information on the internet can come and go, but equivalent information can be found by searching the internet or other appropriate reference source. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/of” unless stated otherwise. Furthermore, use of the term “including” as well as other forms, such as “include”, “includes”, and “included” is not limiting.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

The compounds presented herein may exist as tautomers. Tautomers are compounds that are interconvertible by migration of a hydrogen atom, accompanied by a switch of a single bond and adjacent double bond. In solutions where tautomerization is possible, a chemical equilibrium of the tautomers will exist. The exact ratio of the tautomers depends on several factors, including temperature, solvent, and pH. Some examples of tautomeric pairs include:

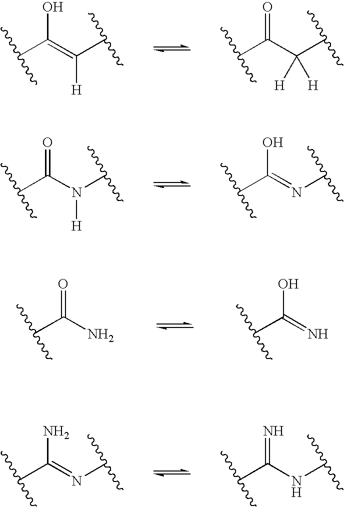

The HDACs are a family including at least eighteen enzymes, grouped in three classes (Class I, II and III). Class I HDACs include, but are not limited to, HDACs 1, 2, 3, and 8. Class I HDACs can be found in the nucleus and are believed to be involved with transcriptional control repressors. Class II HDACs include, but are not limited to, HDACS 4, 5, 6, 7, and 9 and can be found in both the cytoplasm as well as the nucleus. Class III HDACs are believed to be NAD dependent proteins and include, but are not limited to, members of the Sirtuin family of proteins. Non-limiting examples of sirtuin proteins include SIRT1-7. As used herein, the term “selective HDAC” refers to an HDAC inhibitor that does not significantly interact with all three HDAC classes. As used herein, a “Class I selective HDAC” refers to an HDAC inhibitor that interacts with one or more of HDACs 1, 2, 3 or 8, but does not significantly interact with the Class II HDACs (i.e., HDACs 4, 5, 6, 7 and 9).

The term “HDAC modulator” as used herein refers to a compound that has the ability to modulate transcriptional activity.

The term “HDAC inhibitor” as used herein refers to a compound that has the ability to inhibit histone deacetylase activity. This therapeutic class is able to block angiogenesis and cell cycling, and promote apoptosis and differentiation. HDAC inhibitors both display targeted anticancer activity by itself and improve the efficacy of existing agents as well as other new targeted therapies.

The term “subject”, “patient” or “individual” as used herein in reference to individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the mammal is a human.

The terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

As used herein, the terms “cancer treatment”, “cancer therapy” and the like encompasses treatments such as surgery (such as cutting, abrading, ablating (by physical or chemical means or a combination of physical or chemical means), suturing, lasering or otherwise physically changing body tissues and organs), radiation therapy, administration of chemotherapeutic agents and combinations of any two or all of these methods. Combination treatments may occur sequentially or concurrently. Treatments(s), such as radiation therapy and/or chemotherapy, that is administered prior to surgery, is referred to as neoadjuvant therapy. Treatments(s), such as radiation therapy and/or chemotherapy, administered after surgery is referred to herein as adjuvant therapy.

Examples of surgeries that may be used for cancer treatment include, but are not limited to radical prostatectomy, cryotherapy, mastectomy, lumpectomy, transurethral resection of the prostate, and the like.

Many chemotherapeutic agents are known and may operate via a wide variety of modes of action. In some nonlimiting embodiments of the present invention, the chemotherapeutic agent is a cytotoxic agent, an antiproliferative, a targeting agent (such as kinase inhibitors and cell cycle regulators), or a biologic agent (such as cytokines, vaccines, viral agents, and other immunostimulants such as BCG, hormones, monocolonal antibodies and siRNA). The nature of a combination therapy involving administration of a chemotherapeutic agent will depend upon the type of agent being used.

The HDAC inhibitor may be administered in combination with surgery, as an adjuvant, or as a neoadjuvant agent. The HDAC inhibitor may be useful in instances where radiation and/or chemotherapy are indicated, to enhance the therapeutic benefit of these treatments, including induction chemotherapy, primary (neoadjuvant) chemotherapy, and both adjuvant radiation therapy and adjuvant chemotherapy. Radiation and chemotherapy frequently are indicated as adjuvants to surgery in the treatment of cancer. For example, radiation can be used both pre- and post-surgery as components of the treatment strategy for rectal carcinoma. The HDAC inhibitor may be useful following surgery in the treatment of cancer in combination with radiation and/or chemotherapy.

Where combination treatments are contemplated, it is not intended that the HDAC inhibitor be limited by the particular nature of the combination. For example, the HDAC inhibitor may be administered in combination as simple mixtures as well as chemical hybrids. An example of the latter is where the compound is covalently linked to a targeting carrier or to an active pharmaceutical. Covalent binding can be accomplished in many ways, such as, though not limited to, the use of a commercially available cross-linking compound.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that the HDAC inhibitor, and at least one co-agent, are both administered to a patient simultaneously in the form of a single entity or dosage. The term “non-fixed combination” means that the HDAC inhibitor, and at least one co-agent, are administered to a patient as separate entities either simultaneously, concurrently or sequentially with variable intervening time limits, wherein such administration provides effective levels of the two or more compounds in the body of the patient. These also apply to cocktail therapies, e.g. the administration of three or more active ingredients.

As used herein, the terms “co-administration”, “administered in combination with” and their grammatical equivalents or the like are meant to encompass administration of the selected therapeutic agents to a single patient, and are intended to include treatment regimens in which the agents are administered by the same or different route of administration or at the same or different times. In some embodiments, the HDAC inhibitor will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the HDAC inhibitor and the other agent(s) are administered in a single composition. In some embodiments, the HDAC inhibitor and the other agent(s) are admixed in the composition.

The terms “effective amount”, “therapeutically effective amount” or “pharmaceutically effective amount” as used herein, refer to a sufficient amount of at least one agent or compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising the compound as disclosed herein required to provide a clinically significant decrease in a disease. An appropriate “effective” amount in any individual case may be determined using techniques, such as a dose escalation study.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In preferred embodiments, the compounds and compositions described herein are administered orally.

The term “acceptable” as used herein, with respect to a formulation, composition or ingredient, means having no persistent detrimental effect on the general health of the subject being treated.

The term “pharmaceutically acceptable” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutical composition,” as used herein, refers to a biologically active compound, optionally mixed with at least one pharmaceutically acceptable chemical component, such as, though not limited to carriers, stabilizers, diluents, dispersing agents, suspending agents, thickening agents, and/or excipients.

The term “carrier” as used herein, refers to relatively nontoxic chemical compounds or agents that facilitate the incorporation of the compound into cells or tissues.

The term “agonist,” as used herein, refers to a molecule such as the compound, a drug, an enzyme activator or a hormone modulator which enhances the activity of another molecule or the activity of a receptor site.

The term “antagonist,” as used herein, refers to a molecule such as the compound, a drug, an enzyme inhibitor, or a hormone modulator, which diminishes, or prevents the action of another molecule or the activity of a receptor site.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

The term “pharmaceutically acceptable derivative or prodrug” as used herein, refers to any pharmaceutically acceptable salt, ester, salt of an ester or other derivative of a compound, which, upon administration to a recipient, is capable of providing, either directly or indirectly, a pharmaceutically active metabolite or residue thereof. Particularly favored derivatives or prodrugs are those that increase the bioavailability of the compounds of this invention when such compounds are administered to a patient (e.g., by allowing orally administered compound to be more readily absorbed into blood) or which enhance delivery of the parent compound to a biological compartment (e.g., the brain or lymphatic system).

The term “pharmaceutically acceptable salt” as used herein, refers to salts that retain the biological effectiveness of the free acids and bases of the specified compound and that are not biologically or otherwise undesirable. Compounds described herein may possess acidic or basic groups and therefore may react with any of a number of inorganic or organic bases, and inorganic and organic acids, to form a pharmaceutically acceptable salt. These salts can be prepared in situ during the final isolation and purification of the compounds of the invention, or by separately reacting a purified compound in its free base form with a suitable organic or inorganic acid, and isolating the salt thus formed. Examples of pharmaceutically acceptable salts include those salts prepared by reaction of the compound with a mineral or organic acid or an inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, 7-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogen phosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate. Other acids, such as oxalic, while not in themselves pharmaceutically acceptable, may be employed in the preparation of salts useful as intermediates in obtaining the compounds of the invention and their pharmaceutically acceptable acid addition salts. (See for example Berge et al., J. Pharm. Sci. 1977, 66, 1-19.) Further, those compounds described herein which may comprise a free acid group may react with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, N⁺(C₁₋₄ alkyl)₄, and the like. Representative organic amines useful for the formation of base addition salts include ethylamine, diethylamine, ethylenediamine, ethanolamine, diethanolamine, piperazine and the like. It should be understood that SNDX-275 also include the quaternization of any basic nitrogen-containing groups they may contain. Water or oil-soluble or dispersible products may be obtained by such quaternization. See, for example, Berge et al., supra.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “metabolite,” as used herein, refers to a derivative of the compound which is formed when the compound is metabolized.

The term “active metabolite,” as used herein, refers to a biologically active derivative of the compound that is formed when the compound is metabolized.

The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to the compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

Provided herein are methods for treating a patient suffering from diseases associated with abnormal activation of mTOR by administering a therapeutically effective amount of an mTOR inhibitor and a therapeutically effective amount of an HDAC inhibitor. In certain embodiments, the present invention provides methods of treating cancer comprising administering to said individual an effective amount of an mTOR inhibitor and an HDAC inhibitor. In some embodiments, the HDAC and mTOR inhibitors are administered in combination with an additional cancer therapy. In some embodiments, the additional cancer therapy is selected from surgery, radiation therapy, and administration of at least one chemotherapeutic agent. In various embodiments, the administration of the HDAC and mTOR inhibitors occur after surgery. In other embodiments, the administration of the HDAC and mTOR inhibitors occur before surgery. In some embodiments, the cancer is selected from, tumors, leukemias, neoplasms, carcinomas and malignant diseases. In other embodiments, the mTOR inhibitor and HDAC inhibitor are utilized in a method to treat a hyperproliferative disease. In some embodiments, the cancer includes, but is not limited to, brain cancer, breast cancer, lung cancer, ovarian cancer, pancreatic cancer, prostate cancer, renal cancer, colorectal cancer, leukemia, myeloid leukemia, glioblastoma, follicular lymphona, pre-B acute leukemia, chronic lymphocytic B-leukemia, mesothelioma or small cell line cancer. In yet other embodiments, the disorder is a proliferative disease selected from psoriasis, restenosis, autoimmune disease, or atherosclerosis.

Provided herein are methods for degrading, inhibiting the growth of or killing cancer cells comprising contacting the cells with an amount of an mTOR inhibitor and an HDAC inhibitor effective to degrade, inhibit the growth of or kill cancer cells. In some embodiments, the cancer is brain cancer, breast cancer, lung cancer, ovarian cancer, pancreatic cancer, prostate cancer, renal cancer, colorectal cancer, leukemia, myeloid leukemia, glioblastoma, follicular lymphona, pre-B acute leukemia, chronic lymphocytic B-leukemia, mesothelioma or small cell line cancer. In some embodiments, the cancer cells comprise brain, breast, lung, ovarian, pancreatic, prostate, renal, or colorectal cancer cells.

Provided herein are methods of inhibiting tumor size increase, reducing the size of a tumor, reducing tumor proliferation or preventing tumor proliferation in an individual comprising administering to said individual an effective amount of an mTOR inhibitor and an HDAC inhibitor described herein to inhibit tumor size increase, reduce the size of a tumor, reduce tumor proliferation or prevent tumor proliferation. In some embodiments, the tumor occurs in the brain, breast, lung, ovaries, pancreas, prostate, kidney, colon or rectum. In some embodiments, the mTOR inhibitor and HDAC inhibitor are administered in combination with an additional cancer therapy including, but not limited to surgery, radiation therapy, and administration of at least one chemotherapeutic agent. In some embodiments, the composition is administered before surgery. In other embodiments, the composition is administered after surgery.

### HDAC Inhibitors

The HDACs are a family including at least eighteen enzymes, grouped in three classes (Class I, II and III). Class I HDACs include, but are not limited to, HADCs 1, 2, 3, 8 and 11. Class I HDACs can be found in the nucleus and are believed to be involved with transcriptional control repressors. Class II HDACs include, but are not limited to, HDACS 4, 5, 6, 7, and 9 and can be found in both the cytoplasm as well as the nucleus. Class III HDACs are believed to be NAD dependent proteins and include, but are not limited to, members of the Sirtuin family of proteins. Non-limiting examples of sirtuin proteins include SIRT1-7. As used herein, the term “selective HDAC” refers to an HDAC inhibitor that does not substantially interact with all three HDAC classes. The term “Class I Selective HDAC” refers to an HDAC inhibitor that does not substantially interact with Class II or Class III HDACs.

In various embodiments, the HDAC is a non-selective HDAC inhibitor. In specific embodiments, the non-selective HDAC inhibitor is, by way of non-limiting example, N′-hydroxy-N-phenyl-octanediamide (suberoylanilide hydroxamic acid, SAHA), pyroxamide, CBHA, trichostatin A (TSA), trichostatin C, salicylihydroxamic acid (SBHA), azelaic bihydroxamic acid (ABHA), azelaic-1-hydroxamate-9-analide (AAHA), depsipeptide, FK228, 6-(3-chlorophenylureido) carpoic hydroxamic acid (3C1-UCHA), oxamflatin, A-161906, scriptaid, PXD-101, LAQ-824, CHAP, MW2796, LBH589 or MW2996.

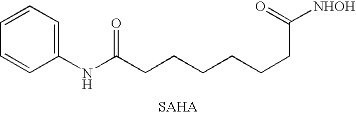

In certain embodiments, the HDAC inhibitor inhibits at least one of HDAC-1, HDAC-2, HDAC-3, HDAC-8, or HDAC-11. In a specific embodiment, the first agent inhibits HDAC-1. In another embodiment, the HDAC inhibitor inhibits HDAC-2. In yet another embodiment, the first agent inhibits HDAC-3. In another embodiment, the HDAC inhibitor inhibits HDAC-8. In still another embodiment, the HDAC inhibitor inhibits HDAC-11. In other embodiments, the HDAC inhibitor inhibits HDAC-1, HDAC-2, HDAC-3 and HDAC-11.

In specific embodiments of the present invention the Class I selective HDAC inhibitor is, by way of non-limiting example, MGCD-0103 (N-(2-amino-phenyl)-4-[(4-pyridin-3-yl-pyrimidin-2-ylamino)-methyl]-benzamide), MS-275 (N-(2-aminophenyl)-4-(N-(pyridin-3-ylmethoxycarbonyl)aminomethyl) benzamide, SNDX-275), spiruchostatin A, SK7041, SK7068 and 6-amino nicotinamides.

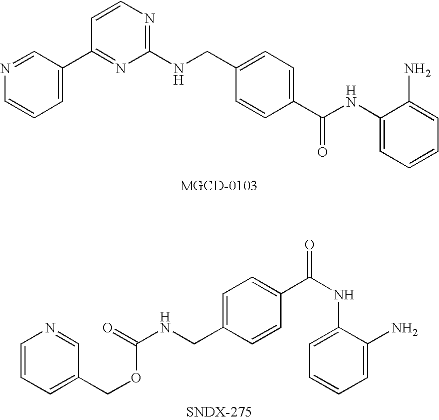

### Synthesis of SNDX-275

SNDX-275 may be obtained by synthesis as described in U.S. Pat. No. 6,174,905 (“U.S. Pat. No. '905”), issued on Jan. 16, 2001. Specifically, the synthesis of SNDX-275 appearing at Example 48 of U.S. Pat. No. '905 is incorporated by reference herein in its entirety.

### Pharmaceutically Acceptable Salts

HDAC inhibitors (e.g., SNDX-275) and mTOR inhibitors may also exist as its pharmaceutically acceptable salts, which may also be useful for treating disorders. For example, the invention provides for methods of treating diseases, by administering pharmaceutically acceptable salts of SNDX-275. The pharmaceutically acceptable salts can be administered as pharmaceutical compositions.

Thus, SNDX-275 can be prepared as pharmaceutically acceptable salts formed when an acidic proton present in the parent compound either is replaced by a metal ion, for example an alkali metal ion, an alkaline earth ion, or an aluminum ion; or coordinates with an organic base. Base addition salts can also be prepared by reacting the free acid form of SNDX-275 with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, the salt forms of the disclosed compounds can be prepared using salts of the starting materials or intermediates.

Further, SNDX-275 can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

### Solvates

HDAC inhibitors (e.g., SNDX-275) and mTOR inhibitors may also exist in various solvated forms, which may also be useful for treating disorders. For example, the invention provides for methods of treating diseases, by administering solvates of SNDX-275. The solvates can be administered as pharmaceutical compositions. Preferably the solvates are pharmaceutically acceptable solvates.

Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of SNDX-275 can be conveniently prepared or formed during the processes described herein. By way of example only, hydrates of SNDX-275 can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Polymorphs

HDAC inhibitors (e.g., SNDX-275) and mTOR inhibitors may also exist in various polymorphic states, all of which are herein contemplated, and which may also be useful for treating disorders. For example, the invention provides for methods of treating diseases, by administering polymorphs of SNDX-275. The various polymorphs can be administered as pharmaceutical compositions.

Thus, SNDX-275 include all crystalline forms, known as polymorphs. Polymorphs include the different crystal packing arrangements of the same elemental composition of the compound. Polymorphs may have different X-ray diffraction patterns, infrared spectra, melting points, density, hardness, crystal shape, optical and electrical properties, stability, solvates and solubility. Various factors such as the recrystallization solvent, rate of crystallization, and storage temperature may cause a single crystal form to dominate.

mTOR Inhibitors

Generally speaking, there are two types of mTOR inhibiting compounds. These are rapamycin analogs and non-rapamycin analogs. Rapamycin has the following structure:

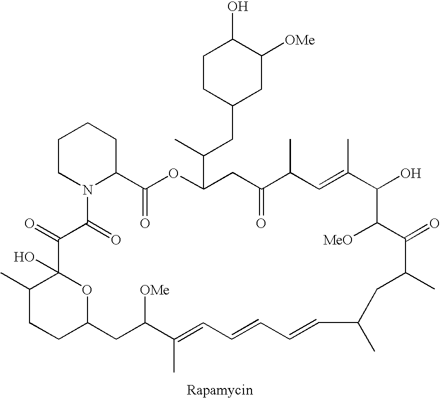

Rapamycin analogs include, but are not limited to, rapamycin (sirolimus or Rapamune (Wyeth)), temsirolimus or CCI-779 (Wyeth, see, U.S. Pat. Nos. 5,362,718 and 6,277,983, the contents of which are incorporated by reference herein in their entirety), everolimus or RAD001 (Novartis), deforolimus or AP23573 (Ariad), AP21967 (Ariad), biolimus (Nobori), AP23102 (Ariad), zotarolimus or ABT 578 (Abbott Labs.), and tacrolimus or Prograf (Astellas).

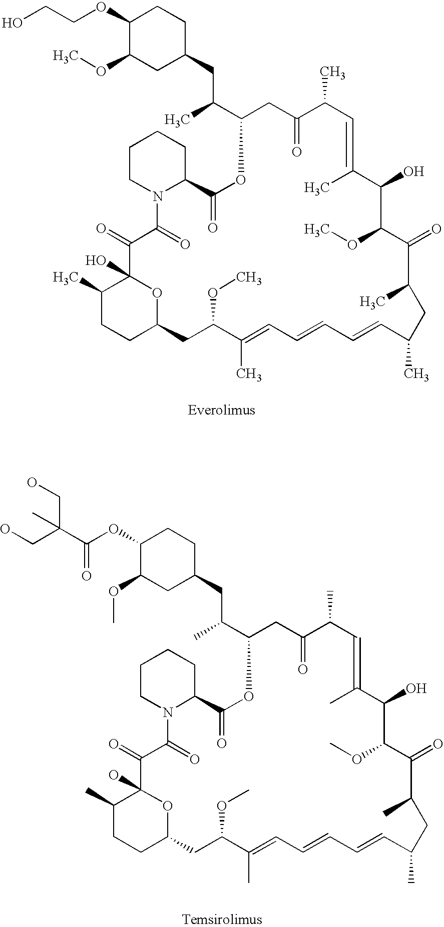

Non-rapamycin analog mTOR inhibiting compounds include, but are not limited to, LY294002, wortmannin, quercetin, myricentin, staurosporine, and ATP competitive inhibitors (see U.S. patent application Ser. Nos. 11/361,213 and 11/361,599, each of which are incorporated by references herein in their entirety).

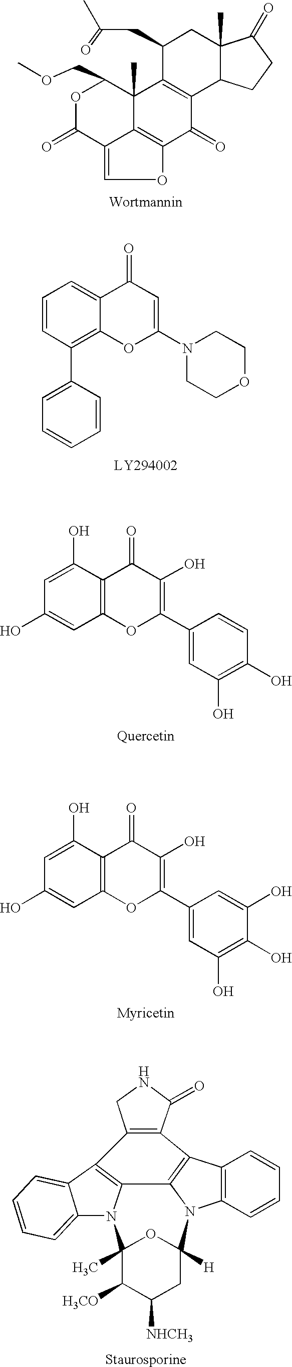

In a specific example, the HDAC inhibitor is MS-275 and the mTOR inhibitor is sirolimus. In another embodiment, the HDAC inhibitor is SAHA and the mTOR inhibitor is everolimus. In other embodiments, the HDAC inhibitor is MS-275 and the mTOR inhibitor is temsirolimus.

In certain embodiments of the present invention, there is provided a method of treating cancer by administering an HDAC inhibitor to a patient, wherein the HDAC inhibitor sensitizes the cancer to the mTOR inhibitor, which is subsequently administered. In some embodiments, the HDAC inhibitor is MS-275 and the mTOR inhibitor is sirolimus.

### Pharmaceutical Compositions

The active pharmaceutical agents of the present invention can be administered alone or as a pharmaceutical composition, thus the invention further provides pharmaceutical compositions and methods of making said pharmaceutical composition. In some embodiments, the pharmaceutical compositions comprise an effective amount of an HDAC inhibitor and an mTOR inhibitor. The pharmaceutical composition may comprise of admixing at least one active ingredient, or a pharmaceutically acceptable salt, prodrug, solvate, polymorph, tautomer or isomer thereof, together with one or more carriers, excipients, buffers, adjuvants, stabilizers, or other materials well known to those skilled in the art and optionally other therapeutic agents. The formulations may conveniently be presented in unit dosage form and may be prepared by any methods well known in the art of pharmacy. The HDAC inhibitor and mTOR inhibitor may be in the same pharmaceutical composition or different pharmaceutical compositions.

Examples of excipients that may be used in conjunction with the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Example of pharmaceutically acceptable carriers that may optionally be used include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

In some embodiments the pharmaceutical compositions comprising an mTOR inhibitor and/or an HDAC inhibitor (e.g., MS-275) are for the treatment of one or more specific disorders. In some embodiments the pharmaceutical compositions are for the treatment of disorders in a mammal, especially a human. In some embodiments the pharmaceutical compositions are for the treatment of cancer such as acute myeloid leukemia, thymus, brain, lung, squamous cell, skin, eye, etc.

### Inhibition of Histone Deacetylase

The invention described herein provides a method of inhibiting histone deacetylase in a cell, comprising contacting a cell in which inhibition of histone deacetylase is desired with an inhibitor of histone deacetylase according to the present invention. Because compounds of the invention inhibit histone deacetylase, they are useful research tools for in vitro study of the role of histone deacetylase in biological processes. In addition, the compounds of the invention selectively inhibit certain isoforms of HDAC.

Measurement of the enzymatic activity of a histone deacetylase can be achieved using known methodologies. For example, Yoshida et al., J. Biol. Chem., 265: 17174-17179 (1990), which is incorporated by reference herein in its entirety, describes the assessment of histone deacetylase enzymatic activity by the detection of acetylated histones in trichostatin A treated cells. Taunton et al., Science, 272: 408-411 (1996), which is incorporated by reference in its entirety, similarly describes methods to measure histone deacetylase enzymatic activity using endogenous and recombinant HDAC-1.

In some embodiments, the histone deacetylase inhibitor interacts with and reduces the activity of all histone deacetylases in the cell. In other embodiments according to this aspect of the invention, the histone deacetylase inhibitor interacts with and reduces the activity of fewer than all histone deacetylases in the cell. In certain other embodiments, the inhibitor interacts with and reduces the activity of one histone deacetylase (e.g., HDAC-1), but does not interact with or reduce the activities of other histone deacetylases (e.g., HDAC-2, HDAC-3, HDAC-4, HDAC-5, HDAC-6, HDAC-7, and HDAC-8). In some embodiments, the histone deacetylase inhibitor of the present invention interacts with, and reduces the enzymatic activity of, a histone deacetylase that is involved in tumorigenesis. In other embodiments, the histone deacetylase inhibitors of the present invention interact with and reduce the enzymatic activity of a fungal histone deacetylase. In some embodiments, SNDX-275 acts as a Class I Selective HDAC inhibitor.

In some embodiments, the compounds and methods of the present invention cause an inhibition of cell proliferation of the contacted cells. The phrase “inhibiting cell proliferation” is used to denote an ability of an inhibitor of histone deacetylase to retard the growth of cells contacted with the inhibitor as compared to cells not contacted. An assessment of cell proliferation can be made by counting contacted and non-contacted cells using a Coulter Cell Counter (Coulter, Miami, Fla.) or a hemacytometer. Where the cells are in a solid growth such as, but not limited to, a solid tumor or organ, an assessment of cell proliferation can be made by measuring the growth with calipers and comparing the size of the growth of contacted cells with non-contacted cells. In some embodiments, growth of cells contacted with the inhibitor is retarded by at least 50% as compared to growth of non-contacted cells. In other embodiments, cell proliferation is inhibited by at least 75%. In still other embodiments, cell proliferation is inhibited by 100% (i.e., the contacted cells do not increase in number). Thus, an inhibitor of histone deacetylase according to the invention that inhibits cell proliferation in a contacted cell may induce the contacted cell to undergo growth retardation, to undergo growth arrest, to undergo programmed cell death (i.e., to apoptose), or to undergo necrotic cell death.

### Methods for Treatment

Described herein are compounds, pharmaceutical compositions and methods for treating a patient suffering from cancer by administering an effective amount of an HDAC inhibitor and an mTOR inhibitor, alone or in combination with one or more additional active ingredients. In some embodiments, the HDAC inhibitor is a Class I Selective HDAC inhibitor. In some embodiments, the HDAC inhibitor is SNDX-275.

In some embodiments, the HDAC inhibitor and mTOR inhibitor are used in combination for the treatment of a hyperproliferative disorder including, but not limited to, hematologic and nonhematologic cancers, cancerous and precancerous skin lesions, leukemias, hyperplasias, fibrosis, angiogenesis, psoriasis, atherosclerosis, and smooth muscle proliferation in the blood vessels.

In some embodiments, the combination therapy is used in the treatment of a malignant disease including, but not limited to, malignant fibrous histiocytoma, malignant mesothelioma, and malignant thymoma.

In some embodiments, the combination therapy is used in wound healing including, but not limited to, healing of wounds associated with radiation therapy.

In some embodiments, the combination therapy is used in the treatment of cancer, tumors, leukemias, neoplasms, or carcinomas, including but not limited to cancer is brain cancer, breast cancer, lung cancer, ovarian cancer, pancreatic cancer, prostate cancer, renal cancer, colorectal cancer, leukemia, myeloid leukemia, glioblastoma, follicular lymphona, pre-B acute leukemia, chronic lymphocytic B-leukemia, mesothelioma or small cell lung cancer. Additional cancers to be treated with the combinations described herein include hematologic and non-hematologic cancers. Hematologic cancer includes multiple myeloma, leukemias, and lymphomas, acute leukemia, acute lymphocytic leukemia (ALL) and acute nonlymphocytic leukemia (ANLL), chronic lymphocytic leukemia (CLL) and chronic myelogenous leukemia (CML). Lymphoma further includes Hodgkin's lymphoma and non-Hodgkin's lymphoma, cutaneous t-cell lymphoma (CTCL) and mantle cell lymphoma (MCL). Non-hematologic cancer includes brain cancer, cancers of the head and neck, lung cancer, breast cancer, cancers of the reproductive system, cancers of the gastro-intestinal system, pancreatic cancer, and cancers of the urinary system, cancer of the upper digestive tract or colorectal cancer, bladder cancer or renal cell carcinoma, and prostate cancer.

In some embodiments, the cancers to treat with the methods and compositions described herein include cancers that are epithelial malignancies (having epithelial origin), and particularly any cancers (tumors) that express EGFR. Non-limiting examples of premalignant or precancerous cancers/tumors having epithelial origin include actinic keratoses, arsenic keratoses, xeroderma pigmentosum, Bowen's disease, leukoplakias, metaplasias, dysplasias and papillomas of mucous membranes, e.g. of the mouth, tongue, pharynx and larynx, precancerous changes of the bronchial mucous membrane such as metaplasias and dysplasias (especially frequent in heavy smokers and people who work with asbestos and/or uranium), dysplasias and leukoplakias of the cervix uteri, vulval dystrophy, precancerous changes of the bladder, e.g. metaplasias and dysplasias, papillomas of the bladder as well as polyps of the intestinal tract. Non-limiting examples of semi-malignant or malignant cancers/tumors of the epithelial origin are breast cancer, skin cancer (e.g., basal cell carcinomas), bladder cancer (e.g., superficial bladder carcinomas), colon cancer, gastro-intestinal (GI) cancer, prostate cancer, uterine cancer, cervical cancer, ovarian cancer, esophageal cancer, stomach cancer, laryngeal cancer and lung cancer.

Additional types of cancers which may be treated using the compositions and methods described herein include: cancers of oral cavity and pharynx, cancers of the respiratory system, cancers of bones and joints, cancers of soft tissue, skin cancers, cancers of the genital system, cancers of the eye and orbit, cancers of the nervous system, cancers of the lymphatic system, and cancers of the endocrine system. These cancers further include cancer of the tongue, mouth, pharynx, or other oral cavity; esophageal cancer, stomach cancer, or cancer of the small intestine; colon cancer or rectal, anal, or anorectal cancer; cancer of the liver, intrahepatic bile duct, gallbladder, pancreas, or other biliary or digestive organs; laryngeal, bronchial, and other cancers of the respiratory organs; heart cancer, melanoma, basal cell carcinoma, squamous cell carcinoma, other non-epithelial skin cancer; uterine or cervical cancer; uterine corpus cancer; ovarian, vulvar, vaginal, or other female genital cancer; prostate, testicular, penile or other male genital cancer; urinary bladder cancer; cancer of the kidney; renal, pelvic, or urethral cancer or other cancer of the genito-urinary organs; thyroid cancer or other endocrine cancer; chronic lymphocytic leukemia; and cutaneous T-cell lymphoma, both granulocytic and monocytic.

Yet other types of cancers which may be treated using the compositions and methods described herein include: adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, liposarcoma, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, and Wilm's tumor.

### Abnormal Cell Growth

In some embodiments, the combination therapy inhibits abnormal cell growth. Methods for inhibiting abnormal cell growth in a mammal comprise administering to the mammal a therapeutically effective amount of the HDAC inhibitor and the mTOR inhibitor in an amount effective to inhibit the abnormal cell growth in the mammal.

In some embodiments, an additional chemotherapeutic is also administered. Many chemotherapeutics are presently known in the art and can be used in combination with the compounds of the invention. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Also described are methods for inhibiting abnormal cell growth in a mammal a therapeutically effective amount of the HDAC inhibitor and the mTOR inhibitor in combination with radiation therapy, wherein the amounts of the HDAC inhibitor and the mTOR inhibitor, in combination with the radiation therapy, is effective in inhibiting abnormal cell growth or treating the hyperproliferative disorder in the mammal. Techniques for administering radiation therapy are known in the art, and these techniques can be used in the combination therapy described herein.

### Treatment Based on Histology of Cancer

Described herein are compounds, pharmaceutical compositions and methods for treating a patient suffering from cancer by administering an effective amount of an HDAC inhibitor and an mTOR inhibitor, alone or in combination with one or more additional active ingredients. In some embodiments, the HDAC inhibitor is a Class I Selective HDAC inhibitor. In some embodiments, the HDAC inhibitor is SNDX-275.

In some embodiments, the cancer is of epithelial origin. Non-limiting examples of cancers of epithelial origin are actinic keratoses, arsenic keratoses, xeroderma pigmentosum, Bowen's disease, leukoplakias, metaplasias, dysplasias and papillomas of mucous membranes, e.g. of the mouth, tongue, pharynx and larynx, precancerous changes of the bronchial mucous membrane such as metaplasias and dysplasias (especially frequent in heavy smokers and people who work with asbestos and/or uranium), dysplasias and leukoplakias of the cervix uteri, vulval dystrophy, precancerous changes of the bladder, e.g. metaplasias and dysplasias, papillomas of the bladder as well as polyps of the intestinal tract. Non-limiting examples of semi-malignant or malignant cancers/tumors of the epithelial origin are breast cancer, skin cancer (e.g., basal cell carcinomas), bladder cancer (e.g., superficial bladder carcinomas), colon cancer, gastro-intestinal (GI) cancer, prostate cancer, uterine cancer, cervical cancer, ovarian cancer, esophageal cancer, stomach cancer, laryngeal cancer and lung cancer.

Cancers of epithelial origin can also be identified by similar histology. Common histological markers for epithelial cancers are mucin 16 (CA125), mucin 1, transmembrane (MUC1), mesothelin, WAP four-disulfide core demain 2 (HE4), kallikrein 6, kallikrein 10, matrix metallopreinase 2, prostasin, osteopontin, tetranectin, and inhibin. Additional histological markers include prostate-specific antigen (PSA), MUC6, IEN, and aneuploidy. Additional examples of histological markers for epithelial cancers include E-cadherin, EZH2, Nectin-4, Her-2, p53, Ki-67, ErbB3, ZEB1 and/or SIP1 expression.

In some embodiments, the cancer is a hematological cancer. Non-limiting examples of hematological cancers include lymphoma (including, but not limited to, Hodgkin's lymphoma, diffuse large b-cell lymphoma (DLBCL) also know as immunoblastic lymphoma, aggressive lymphomas also known as intermediate and high grade lymphomas, indolent lymphomas also known as low grade lymphomas, mantle cell lymphoma, follicular lymphoma), leukemia, acute promyelocytic leukemia, acute myeloideleukaemia, chronic myeloide leukaemia, chronic lymphatic leukaemia, Hodgkin's disease, multiple myeloma, myelodysplasia, myeloproliferative disease, and refractory anemia.

Hematological cancers can also be identified by similar histology. Common histological markers for hematological cancers are tumor-antigens, M34, antibodies, cancer antigens, CA15-3, carcinoembryonic antigen, CA125, cytokeratins, hMAM, MAGE, pancytokeratins, and HLA Class I or Class II antigens such as HLA-DR and HLA-D, MB, MT, MTe, Te, and SB. Additional examples of histological markers for B-cell malignancies include CD5, CD6, CD10, CD19, CD20, CD21, CD22, CD23, CD24, CD25, CD26, CD28, CD30, CD32, CD35, CD37, CD38, CD39, CD40, CD43, CD45RO, CD45RA, CD45RB, CD49B, CD49C, CD49D, CD50, CD52, CD57, CD62L, CD69, CD70, CD72, CD73, CD74, CD75, CD77, CD79α,β, CD80, CD83, CDW84, CD86, CD89, CD97, CD98, CD119, CDW121B, CD122, CD124, CD125, CD126, CD127, CD130, CD132, CD135, CDW137, CD171, CD179A, CD179B, CD180, CD183, CDW197, CD200, CDW210, CD213A1 and CD213A2. Examples of histological markers for T-cell malignancies include CD4, CD8, CD5, CD2, CD25, CD26, CD28, CD27, CD30, CD37, CD38, CD45RO, CD45RA, CD45RB, CD49A, CD49E, CD49F, CD50, CD52, CD56, CD57, CD62L, CD69, CD70, CD73, CD89, CD90, CD94, CD96, CD97, CD98, CD101, CD107A, CD107B, CD109, CD121A, CD122, CD124, CDW128, CD132, CD134, CDW137, CD148, CD152, CD153, CD154, CD160, CD161, CD165, CD166, CD171, CD178, CDW197, CDW210, CD212, CDW217, CD223, CD226, CD231, CD245 and CD247.

In some embodiments, the cancer is a neuroendocrine cancer. Non-limiting examples of neuroendocrine cancers include lung and pancreatic cancers as well as neuroendocrine tumors of the digestive system. More specifically, these types of cancer may be called gastrinoma, insulinoma, glucagonoma, vasoactive intestinal peptideoma (VIPoma), PPoma, somatostatinoma, CRHoma, calcitoninoma, GHRHoma, ACTHoma, and GRFoma. Additional examples of neuroendocrine cancers include medullary carcinoma of the thyroid, Merkel cell cancer, small-cell lung cancer (SCLC), large-cell neuroendocrine carcinoma of the lung, neuroendocrine carcinoma of the cervix, Multiple Endocrine Neoplasia type 1 (MEN-1 or MEN1), Multiple Endocrine Neoplasia type 2 (MEN-2 or MEN2), neurofibromatosis type 1, tuberous sclerosis, von Hippel-Lindau (VHL) disease, neuroblastoma, pheochromocytoma (phaeochromocytoma), paraganglioma, neuroendocrine tumor of the anterior pituitary, and Camey's complex.

Neuroendocrine cancers can also be identified by similar histology. Common histological markers for neuroendocrine cancers are hormone markers, chromogranin A (CgA), urine 5-hydroxy indole acetic acid (5-HIAA) (grade C), neuron-specific enolase (NSE, gamma-gamma dimer), synaptophysin (P38), N-terminally truncated variant of heat shock protein 70 (Hsp 70), CDX-2, neuroendocrine secretory protein-55, and blood serotonin.

Other histological markers are known in the art provide the ability to potentially identify and distinguish cancer cells from normal cells or within different types of cancers or malignancies.

### Modes of Administration

Administration of the actives and compositions described herein can be effected by any method that enables delivery of the actives to the site of action. These methods include oral routes, intraduodenal routes, parenteral injection (including intravenous, subcutaneous, intraperitoneal, intramuscular, intravascular or infusion), topical, intrapulmonary, rectal administration, by implant, by a vascular stent impregnated with the compound, and other suitable methods commonly known in the art. For example, actives described herein can be administered locally to the area in need of treatment. This may be achieved by, for example, but not limited to, local infusion during surgery, topical application, e.g., cream, ointment, injection, catheter, or implant, said implant made, e.g., out of a porous, non-porous, or gelatinous material, including membranes, such as sialastic membranes, or fibers. The administration can also be by direct injection at the site (or former site) of a tumor or neoplastic or pre-neoplastic tissue. Those of ordinary skill in the art are familiar with formulation and administration techniques that can be employed with the actives and methods of the invention, e.g., as discussed in Goodman and Gilman, The Pharmacological Basis of Therapeutics, (current edition); Pergamon; and Remington's, Pharmaceutical Sciences (current edition), Mack Publishing Co., Easton, Pa.

The formulations include those suitable for oral, parenteral (including subcutaneous, intradermal, intramuscular, intravenous, intraarticular, intramedullary, intracardiac, intrathecal, intraspinal, intracapsular, subcapsular, intraorbital, intratracheal, subcuticular, intraarticular, subarachnoid, and intrasternal), intraperitoneal, transmucosal, transdermal, rectal and topical (including dermal, buccal, sublingual, intranasal, intraocular, and vaginal) administration although the most suitable route may depend upon for example the condition and disorder of the recipient. The formulations may conveniently be presented in unit dosage form and may be prepared by any of the methods well known in the art of pharmacy. All methods include the step of bringing into association the active ingredient with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders (e.g., povidone, gelatin, hydroxypropylmethyl cellulose), inert diluents, preservative, disintegrant (e.g., sodium starch glycolate, cross-linked povidone, cross-linked sodium carboxymethyl cellulose) or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. Tablets may optionally be provided with an enteric coating, to provide release in parts of the gut other than the stomach. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

Pharmaceutical preparations may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, biocide, bacteriostats and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable lipophilic solvents or vehicles include fatty oils such as sesame oil, or synthetic fatty acid esters, such as ethyl oleate or triglycerides, or liposomes or other microparticulate systems may be used to target the compound to blood components or one or more organs. The concentration of the active ingredient in the solution may vary widely. Typically, the concentration of the active ingredient in the solution is from about 1 ng/ml to about 10 μg/ml, for example from about 10 ng/ml to about 1 μg/ml. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions

Pharmaceutical preparations may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastimes, or gels formulated in conventional manner. Such compositions may comprise the active ingredient in a flavored basis such as sucrose and acacia or tragacanth.

Pharmaceutical preparations may also be formulated in rectal compositions such as suppositories or retention enemas, e.g., containing conventional suppository bases such as cocoa butter, polyethylene glycol, or other glycerides.

Pharmaceutical preparations may be administered topically, that is by non-systemic administration. This includes the application of the compositions externally to the epidermis or the buccal cavity and the instillation of such compound into the ear, eye and nose, such that the compound does not significantly enter the blood stream. In contrast, systemic administration refers to oral, intravenous, intraperitoneal and intramuscular administration.

Pharmaceutical preparations suitable for topical administration include liquid or semi-liquid preparations suitable for penetration through the skin to the site of inflammation such as gels, liniments, lotions, creams, ointments or pastes, suspensions, powders, solutions, spray, aerosol, oil, and drops suitable for administration to the eye, ear or nose. Alternatively, a formulation may comprise a patch or a dressing such as a bandage or adhesive plaster impregnated with active ingredients and optionally one or more excipients or diluents. The amount of active ingredient present in the topical formulation may vary widely. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Formulations suitable for topical administration in the mouth include lozenges comprising the active ingredient in a flavored basis, usually sucrose and acacia or tragacanth; pastimes comprising the active ingredient in an inert basis such as gelatin and glycerin, or sucrose and acacia; and mouthwashes comprising the active ingredient in a suitable liquid carrier.

Formulations suitable for topical administration to the eye also include eye drops wherein the active ingredient is dissolved or suspended in a suitable carrier, especially an aqueous solvent for the active ingredient.

Pharmaceutical preparations for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

In various embodiments, SNDX-275 may be prepared as a free base or a pharmaceutically acceptable salt, solvate, polymorph, ester, tautomer or prodrug thereof. Also described, are pharmaceutical compositions comprising SNDX-275 or a pharmaceutically acceptable salt, solvate, polymorph, ester, tautomer or prodrug thereof. The compounds and compositions described herein may be administered either alone or in combination with pharmaceutically acceptable carriers, excipients or diluents, in a pharmaceutical composition, according to standard pharmaceutical practice. In some embodiments, SNDX-275 is formulated as a solid dosage form, such as a tablet, capsule, caplet, powder, etc. In some embodiments, SNDX-275 is formulated as a tablet, wherein the tablet contains from about 0.1 to about 12 mg, e.g. about 1, 2, 3, 4, 5, 6, 7, 8, 9, 10, 11 or 12 mg. In some embodiments, SNDX-275 is formulated as a tablet containing 2, 3, 4, 5, 7 or 10 mg of SNDX-275.

### Formulations

The actives or compositions described herein can be delivered in a vesicle, e.g., a liposome (see, for example, Langer, Science 1990, 249, 1527-1533; Treat et al., Liposomes in the Therapy of Infectious Disease and Cancer, Lopez-Bernstein and Fidler, Ed., Liss, N.Y., pp. 353-365, 1989). The actives and pharmaceutical compositions described herein can also be delivered in a controlled release system. In some embodiments, a pump may be used (see, Sefton, 1987, CRC Crit. Ref. Biomed. Eng. 14:201; Buchwald et al. Surgery, 1980 88, 507; Saudek et al. N. Engl. J. Med. 1989, 321, 574. Additionally, a controlled release system can be placed in proximity of the therapeutic target. (See, Goodson, Medical Applications of Controlled Release, 1984, Vol. 2, pp. 115-138). The pharmaceutical compositions described herein can also contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions, and such compositions may contain one or more agents selected from, by way of non-limiting example, sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be un-coated or coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate may be employed as appropriate. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

Pharmaceutical compositions may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening agents, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Pharmaceutical compositions may be in the form of a sterile injectable aqueous solution. Among the acceptable vehicles and solvents that may be employed are water, Ringer's solution and isotonic sodium chloride solution. The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion. The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump. The pharmaceutical compositions may be in the form of a sterile injectable aqueous or oleaginous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Pharmaceutical compositions may also be administered in the form of suppositories for rectal administration of the drug. These compositions can be prepared by mixing the inhibitors with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound or composition of the invention can be used. As used herein, topical application can include mouth washes and gargles.

Pharmaceutical compositions may be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

### HDAC Inhibitor Doses

In some embodiments, about 0.5 to about 30 mg of the HDAC inhibitor is administered to the patient. In some embodiments, about 1 to about 8, about 2 to about 6, about 2, about 4, about 6 or about 8 mg of SNDX-275 is administered to the patient, especially where such administration is oral administration. In some embodiments, the administration may be repeated, e.g. on a twice weekly (2×weekly, semiweekly) schedule, a weekly schedule, a biweekly schedule, a monthly schedule, etc. In some embodiments, the HDAC inhibitor is administered on a weekly schedule for 1, 2, 3, 4, 5, 6 or more weeks. In some embodiments, the HDAC inhibitor is administered on a weekly schedule for 1, 2, 3, 4, 5 or 6 or more weeks, followed by a period in which no HDAC inhibitor is administered (wash-out period), which may be 1, 2, 3, 4 or more weeks. In some embodiments, the wash-out period is from about 1 day to about 3 weeks, or about 3 days to about 1 week, or about 1 week to about 2 weeks, or about 2 weeks to about 3 weeks. In some embodiments, the HDAC inhibitor is administered weekly for 2 weeks, followed by a 1, 2 or 3 week wash-out period. In some embodiments, the HDAC inhibitor is administered weekly for 3 weeks, followed by a 1, 2 or 3 week wash-out period. In some embodiments, the HDAC inhibitor is administered weekly for 4 weeks, followed by a 1, 2 or 3 week wash-out period. In some embodiments, the HDAC inhibitor is administered on a weekly schedule for 1, 2, 3, 4, 5, 6 or more weeks. In some embodiments, the HDAC inhibitor is administered on a 2×weekly schedule for 1, 2, 3, 4, 5 or 6 or more weeks, followed by a period in which no HDAC inhibitor is administered (wash-out period), which may be 1, 2, 3, 4 or more weeks. In some embodiments, the HDAC inhibitor is administered 2×weekly for 2 weeks, followed by a 1, 2 or 3 week wash-out period. In some embodiments, the HDAC inhibitor is administered 2×weekly for 3 weeks, followed by a 1, 2 or 3 week wash-out period. In some embodiments, the HDAC inhibitor is administered 2×weekly for 4 weeks, followed by a 1, 2 or 3 week wash-out period. In some embodiments, the HDAC inhibitor is administered on a biweekly schedule. In some embodiments, biweekly dosing is repeated 1, 2, 3, 4, 5, 6 or more times, followed by a period of wash-out. In some embodiments, the HDAC inhibitor is administered on a biweekly schedule for 1, 2, 3, 4, 5 or 6 or more biweeks, followed by a wash-out period of 1, 2, 3, 4 or more weeks. In some embodiments, the HDAC inhibitor is administered biweekly for 2 biweeks, followed by a 1, 2 or 3 week wash-out period. In some embodiments, the HDAC inhibitor is administered biweekly for 3 biweeks, followed by a 1, 2 or 3 week wash-out period. In some embodiments, the HDAC inhibitor is administered weekly for 4 biweeks, followed by a 1, 2 or 3 week wash-out period. In some embodiments, the HDAC inhibitor is administered on a biweekly schedule for 1, 2, 3, 4, 5, 6 or more biweeks.

In some embodiments, SNDX-275 is administered orally in a dosage range of about 2 to about 10, about 2 to about 8 or about 2 to about 6 mg/m². In some embodiments, SNDX-275 is administered to the patient orally at a dosage of about 2, about 4, about 5 or about 6 mg/m². At these dosages, SNDX-275 is administered less frequently than once per day. In some embodiments, the SNDX-275 is administered less frequently than once per week. In some embodiments, the SNDX-275 is administered orally twice per week for at least a week. In some embodiments, SNDX-275 is administered once per week for at least two weeks. In some embodiments, SNDX-275 is administered at least twice every other week. In some embodiments, the administered SNDX-275 produces an area under the plasma concentration curve (AUC) in the patient of about 100 to about 800 ng·h/mL. In some embodiments, the Cₘₐₓ for SNDX-275 is about 1 to about 100 ng/mL. In some embodiments, Tₘₐₓ is achieved from 0.5 to 24 hours after administration of SNDX-275. The treated patient is generally suffering from cancer e.g. a solid tumor cancer or a leukemia.

In some embodiments, SNDX-275 is administered orally to a cancer patient. The cancer may be either a solid tumor or a leukemia. In some embodiments, the administration occurs on a cycle comprising a dosing period and a wash-out period. In some embodiments, the dosing period is biweekly, weekly or 2×weekly. In some embodiments, the oral dose administered is about 1 to 10, about 2 to 8 or about 2 to 6 mg/m² of SNDX-275. In some embodiments, the oral dose is 2, 4, 5, 6, 8 or 10 mg/m² of SNDX-275. In some embodiments, the oral dose of SNDX-275 is 2, 4, 6, 8 or 10 mg/m² of SNDX-275 administered on a 2×weekly schedule, after which the cycle may be repeated. In some embodiments, the oral dose of SNDX-275 administered is 2 mg/m² administered on a 2×weekly schedule, after which the cycle may be repeated. In some embodiments, the oral dose of SNDX-275 administered is 2, 4, 6, 8 or 10 mg/m² on a 2×weekly schedule for 1, 2, 3, 4, 5 or 6 weeks, followed by a 1, 2, 3 or 4 week washout period, after which the cycle may be repeated. In some embodiments, the oral dose of SNDX-275 administered is 2 mg/m² on a 2×weekly schedule for 1, 2, 3, 4, 5 or 6 weeks, followed by a 1, 2, 3 or 4 week washout period, after which the cycle may be repeated. In some embodiments, the oral dose of SNDX-275 administered is 2, 4, 5, 6, 8 or 10 mg/m² of SNDX-275 on a weekly schedule for 1, 2, 3, 4, 5 or 6 weeks, followed by a 1, 2, 3 or 4 week washout period, after which the cycle may be repeated. In some embodiments, the oral dose of SNDX-275 administered is 2 mg/m², 4 mg/m² or 5 mg/m² on a weekly schedule for 1, 2, 3, 4, 5 or 6 weeks, followed by a 1, 2, 3 or 4 week washout period, after which the cycle may be repeated. In some embodiments, the oral dose of SNDX-275 administered is 2, 4, 5, 6, 8 or 10 mg/m² on a biweekly schedule of about 1, 2, 3, 4, 5 or 6 biweeks, followed by a wash-out period of about 1, 2, 3 or 4 weeks, after which the cycle may be repeated. In some embodiments, the oral dose of SNDX-275 administered is 2, 4, 5 or 6 mg/m² on a biweekly schedule of about 1, 2, 3, 4, 5 or 6 biweeks, followed by a wash-out period of about 1, 2, 3 or 4 weeks, after which the cycle may be repeated.

In some embodiments, suitable dosages of SNDX-275 are total weekly dosages of between about 0.25 to about 10 mg/m². They can be administered in various cycles: once weekly at a dose of about 2 to 10 mg; twice weekly at a dose of about 0.5 to about 2 mg; once every other week (biweekly) at a dose of about 2 to 12 mg; three times monthly at a dose of about 2 to 10 mg; four times per six weeks (e.g. four weeks on and two weeks off) at 2 to 10 mg, two times monthly (e.g. 2 weeks on and 2 weeks off) at a dose of 2 to 10 mg.

In some embodiments, so called “flat” dosing of SNDX-275 may be employed. A flat dose is a particular mass of SNDX-275: that is neither the mass nor the surface area of the patient are taken into account when determining the dose. Suitable flat doses contemplated herein are about 0.25, 0.5, 0.75, 1, 2, 3, 4, 5, 6, 7, 8, 9, 10, 11 or 12 mg of SNDX-275 per dose. Particular flat doses contemplated herein are 3, 5, 7 and 10 mg of SNDX-275 per dose. Such doses may be administered on one of dosing schedules described herein. In some embodiments, a dose of about 0.25, 0.5, 0.75, 1, 2, 3, 4, 5, 6, 7, 8, 9, 10, 11 or 12 mg of SNDX-275 per dose is administered on a twice-weekly, weekly (once per week) or biweekly (once every other week) dosing schedule, optionally with a rest period built in after a certain number of dosing cycles. In some embodiments, the dosing schedule is weekly and SNDX-275 is administered at a dose of about 1-12 mg (e.g. about 2, 3, 4, 5, 6, 7, 8, 9 or 10 mg) once a week for two weeks, followed by a rest period (i.e. no chemotherapy) of one, two or three weeks. In some embodiments, the dosing schedule is weekly and SNDX-275 is administered at a dose of about 1-12 mg (e.g. about 2, 3, 4, 5, 6, 7, 8, 9 or 10 mg) once a week for three weeks, followed by a rest period of one, two or three weeks. In some embodiments, the dosing schedule is weekly and SNDX-275 is administered at a dose of about 1-12 mg (e.g. about 2, 3, 4, 5, 6, 7, 8, 9 or 10 mg) once a week for four weeks, followed by a rest period of one, two or three weeks. In some embodiments, the dosing schedule is twice weekly (2×weekly) and SNDX-275 is administered at a dose of about 0.25 to about 8 mg (e.g. about 0.25, 0.5, 0.75, 1, 2, 3, 4, 5 or 6 mg) twice a week for two weeks, followed by a rest period (i.e. no chemotherapy) of one, two or three weeks. In some embodiments, the dosing schedule is 2×weekly and SNDX-275 is administered at a dose of about 0.25 to about 8 mg (e.g. about 0.25, 0.5, 0.75, 1, 2, 3, 4, 5 or 6 mg) twice a week for three weeks, followed by a rest period of one, two or three weeks. In some embodiments, the dosing schedule is 2×weekly and SNDX-275 is administered at a dose of about 0.25 to about 8 mg (e.g. about 0.25, 0.5, 0.75, 1, 2, 3, 4, 5 or 6 mg) twice a week for four weeks, followed by a rest period of one, two or three weeks. In some embodiments, the dosing schedule is every other week (biweekly) and SNDX-275 is administered at a dose of about 2-12 mg (e.g. about 2, 3, 4, 5, 6, 7, 8, 9 or 10 mg) once a biweek (once every other week).

In some embodiments, the total dosage range is about 1 mg to about 12 mg/m² per biweek. In some embodiments, the total dosage range is about 1 mg to about 12 mg/m² per week. In some embodiments, a total dosage will range from about 2 to about 24 mg/m² per month.

In some embodiments, the method of treating cancer in a patient comprises administering to the patient a first dose of 10 mg SNDX-275 during a first biweek of a biweekly dosing schedule and a second dose of 10 mg of SNDX-275 during a second biweek of the biweekly dosing cycle, wherein the biweekly dosing schedule comprises at least two consecutive biweeks. In some embodiments, the first dose of SNDX-275 is administered on day 1 to day 4 of the first biweek and the second dose of SNDX-275 is administered on day 1 to day 4 of the second biweek. In some embodiments, the first dose of SNDX-275 is administered on day 1 to day 3 of the first biweek and the second dose of SNDX-275 is administered on day 1 to day 3 of the second biweek. In some embodiments, the first dose of SNDX-275 is administered on day 1 of the first biweek and the second dose of SNDX-275 is administered on day 1 of the second biweek. In some embodiments, the method further comprises administering to the patient at least one lower dose, including but not limited to a 5 mg dose, of SNDX-275 after the end of the biweekly dosing cycle schedule. In some embodiments, the method further comprises detecting a drug-related toxicity in the patient and subsequently administering to the patient a reduced dose of SNDX-275. In some embodiments, the reduced dose is 5 mg of SNDX-275 per dose. In some embodiments, the reduced dose is administered to the patient on a biweekly dosing schedule, wherein a first dose of 5 mg of SNDX-275 is administered to the patient during the first biweek and a second dose of 5 mg of SNDX-275 is administered to the patient during the second biweek. In some embodiments, the first dose of SNDX-275 is administered on day 1 to day 4 of the first biweek and the second dose of SNDX-275 is administered on day 1 to day 4 of the second biweek. In some embodiments, the first dose of SNDX-275 is administered on day 1 to day 3 of the first biweek and the second dose of SNDX-275 is administered on day 1 to day 3 of the second biweek. In some embodiments, the first dose of SNDX-275 is administered on day 1 of the first biweek and the second dose of SNDX-275 is administered on day 1 of the second biweek. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments meet the foregoing and additional needs by providing a method of treating cancer in a patient, comprising administering to the patient at least one dose of 10 mg of SNDX-275 and at least one subsequent dose of 5 mg of SNDX-275. In some embodiments, the method further comprises, after administering the 10 mg of SNDX-275 to the patient, detecting a drug-related toxicity in the patient, and subsequently administering the 5 mg dose of SNDX-275 to the patient. In some embodiments, the 10 mg dose of SNDX-275 is administered as part of a biweekly dosing schedule, wherein a first dose of 10 mg is administered during a first biweek and optionally a second dose of 10 mg is administered during a second biweek. In some embodiments, the 10 mg dose of SNDX-275 is administered as part of a biweekly dosing schedule, wherein a first dose of 10 mg of SNDX-275 is administered during the first biweek, a drug-related toxicity is then detected, and a second dose of 5 mg of SNDX-275 is administered during the second biweek. In some embodiments, the mean area under the plasma concentration curve of SNDX-275 is about 100 ng·h/mL to about 400 ng·h/mL. In some embodiments, the mean maximum plasma concentration of SNDX-275 is about 1 to about 60 ng/mL. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments meet the foregoing needs and provide related advantages by providing a method of treating cancer in a patient, comprising administering to the patient a first dose of 5 mg SNDX-275 during a first biweek of a biweekly dosing schedule and a second dose of 5 mg of SNDX-275 during a second biweek of the biweekly dosing cycle, wherein the biweekly dosing schedule comprises at least two consecutive biweeks. In some embodiments, the first dose of SNDX-275 is administered on day 1 to day 4 of the first biweek and the second dose of SNDX-275 is administered on day 1 to day 4 of the second biweek. In some embodiments, the first dose of SNDX-275 is administered on day 1 to day 3 of the first biweek and the second dose of SNDX-275 is administered on day 1 to day 3 of the second biweek. In some embodiments, the first dose of SNDX-275 is administered on day 1 of the first biweek and the second dose of SNDX-275 is administered on day 1 of the second biweek. In some embodiments, the mean area under the plasma concentration curve of SNDX-275 is about 150 ng·h/mL to about 350 ng·h/mL. In some embodiments, the mean maximum plasma concentration of SNDX-275 is about 1 to about 50 ng/mL. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments meet the foregoing and additional needs by providing a method of treating cancer in a patient, comprising administering to the patient a first dose of 7 mg SNDX-275 during a first biweek of a biweekly dosing schedule and a second dose of 7 mg of SNDX-275 during a second biweek of the biweekly dosing cycle, wherein the biweekly dosing schedule comprises at least two consecutive biweeks. In some embodiments, the first dose of SNDX-275 is administered on day 1 to day 4 of the first biweek and the second dose of SNDX-275 is administered on day 1 to day 4 of the second biweek. In some embodiments, the first dose of SNDX-275 is administered on day 1 to day 3 of the first biweek and the second dose of SNDX-275 is administered on day 1 to day 3 of the second biweek. In some embodiments, the first dose of SNDX-275 is administered on day 1 of the first biweek and the second dose of SNDX-275 is administered on day 1 of the second biweek. In some embodiments, the mean area under the plasma concentration curve of SNDX-275 is about 100 ng·h/mL to about 400 ng·h/mL. In some embodiments, the mean maximum plasma concentration of SNDX-275 is about 1 to about 60 ng/mL. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

The foregoing and additional needs are met by embodiments that provide a method of treating cancer in a patient, comprising administering to the patient a first dose of 3 mg SNDX-275 during a first biweek of a biweekly dosing schedule and a second dose of 3 mg of SNDX-275 during a second biweek of the biweekly dosing cycle, wherein the biweekly dosing schedule comprises at least two consecutive biweeks. In some embodiments, the first dose of SNDX-275 is administered on day 1 to day 4 of the first biweek and the second dose of SNDX-275 is administered on day 1 to day 4 of the second biweek. In some embodiments, the first dose of SNDX-275 is administered on day 1 to day 3 of the first biweek and the second dose of SNDX-275 is administered on day 1 to day 3 of the second biweek. In some embodiments, the first dose of SNDX-275 is administered on day 1 of the first biweek and the second dose of SNDX-275 is administered on day 1 of the second biweek. In some embodiments, the mean area under the plasma concentration curve of SNDX-275 is about 100 ng·h/mL to about 350 ng·h/mL. In some embodiments, the mean maximum plasma concentration of SNDX-275 is about 1 to about 50 ng/mL. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

The foregoing and additional needs are met by embodiments that provide a method of treating cancer in patient, comprising administering a first dose of from 2 to 6 mg/m² of SNDX-275 on a first day of an at least 28-day dosing cycle, a second dose of from 2 to 6 mg/m² of SNDX-275 on a second day of the at least 28-day dosing cycle and a third dose of from 2 to 6 mg/m² on a third day of the at least 28-day dosing cycle. In some embodiments, the first dose of SNDX-275 is 2 mg/m². In some embodiments, the second dose of SNDX-275 and the third dose of SNDX-275 are each 2 mg/m². In some embodiments, the first dose of SNDX-275 is 4 mg/m². In some embodiments, the second dose of SNDX-275 and the third dose of SNDX-275 are each 4 mg/m². In some embodiments, the first dose of SNDX-275 is 6 mg/m². In some embodiments, the second dose of SNDX-275 and the third dose of SNDX-275 are each 6 mg/m². In some embodiments, the first dose of SNDX-275 is administered on day 1 to day 7 of the at least 28-day dosing cycle and the second dose of SNDX-275 and the third dose of SNDX-275 are each administered on day 8 to day 28 of the at least 28-day dosing cycle. In some embodiments, the first dose of SNDX-275 is administered on day 1 to day 7 of the at least 28-day dosing cycle and the second dose of SNDX-275 and the third dose of SNDX-275 are each administered on day 8 to day 21 of the at least 28-day dosing cycle. In some embodiments, the first dose of SNDX-275 is administered on day 1 to day 4 of the at least 28-day dosing cycle, the second dose of SNDX-275 is administered on day 8 to day 11 of the at least 28-day dosing cycle and the third dose of SNDX-275 is administered on day 15 to day 18 of the at least 28-day dosing cycle. In some embodiments, the first dose of SNDX-275 is administered on day 1 to day 3 of the at least 28-day dosing cycle, the second dose of SNDX-275 is administered on day 8 to day 10 of the at least 28-day dosing cycle and the third dose of SNDX-275 is administered on day 15 to day 17 of the at least 28-day dosing cycle. In some embodiments, the first dose of SNDX-275 is administered on day 1 of the at least 28-day dosing cycle, the second dose of SNDX-275 is administered on day 8 of the at least 28-day dosing cycle and the third dose of SNDX-275 is administered on day 15 of the at least 28-day dosing cycle. In some embodiments, the mean area under the plasma concentration curve of SNDX-275 is about 100 ng·h/mL to about 350 ng·h/mL. In some embodiments, the mean maximum plasma concentration of SNDX-275 is about 1 to about 50 ng/mL. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments provided herein meet the foregoing and additional needs by providing a method of treating cancer in a patient, comprising administering to the patient two doses of about 2 to about 10 mg/m² each of SNDX-275 over the course of a 4 week treatment cycle, wherein a first dose of SNDX-275 is administered during week 1, a second dose of SNDX-275 is administered during week 2, and no dose of SNDX-275 is administered during each of weeks 3 and 4. In some embodiments, the first dose is about 2 mg/m². In some embodiments, the second dose is about 2 mg/m². In some embodiments, the first dose is about 4 mg/m². In some embodiments, the second dose is about 4 mg/m². In some embodiments, the first dose is about 6 mg/m². In some embodiments, the second dose is about 6 mg/m². In some embodiments, the second dose is about 8 mg/m². In some embodiments, the second dose is about 8 mg/m². In some embodiments, the mean area under the plasma concentration curve of SNDX-275 is about 150 ng·h/mL to about 350 ng·h/mL. In some embodiments, the mean maximum plasma concentration of SNDX-275 is about 1 to about 50 ng/mL. In some embodiments, the mean time to maximum plasma concentration of SNDX-275 is about 1.5 to about 6 hours. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments herein provide a method of treating cancer in a patient, comprising administering to the patient four doses of about 2 to about 10 mg/m² each of SNDX-275 over the course of a 6 week treatment cycle, wherein a first dose of SNDX-275 is administered during week 1, a second dose of SNDX-275 is administered during week 2, a third dose of SNDX-275 is administered during week 3, a fourth dose is administered during week 4, and no dose of SNDX-275 is administered during each of weeks 5 and 6. In some embodiments, the first dose is about 2 mg/m². In some embodiments, each of the second, third and fourth doses are about 2 mg/m². In some embodiments, the first dose is about 4 mg/m². In some embodiments, each of the second, third and fourth doses are about 4 mg/m². In some embodiments, the first dose is about 6 mg/m². In some embodiments, each of the second, third and fourth doses are about 6 mg/m². In some embodiments, the first dose is about 8 mg/m². In some embodiments, each of the second, third and fourth doses are about 8 mg/m². In some embodiments, the second dose is about 10 mg/m². In some embodiments, each of the second, third and fourth doses are about 10 mg/m². In some embodiments, the mean area under the plasma concentration curve of SNDX-275 is about 300 ng·h/mL to about 350 ng·h/mL. In some embodiments, the mean maximum plasma concentration of SNDX-275 is about 40 to about 60 ng/mL. In some embodiments, the mean time to maximum plasma concentration of SNDX-275 is about 0.5 to about 6 hours. In some embodiments, SNDX-275 is administered orally. In some embodiments, SNDX-275 is administered orally in the form of one or more tablets. In some embodiments, SNDX-275 is administered orally in the form of 0.5, 1, 2, 3, 4, 5, 6, 7, 8, 9 or 10 mg tablets or a suitable combination of 2 or more thereof.

Some embodiments provide a method of treating cancer in a patient, comprising administering a first dose of a composition comprising 2-10 mg/m² of SNDX-275 on day 1 and administering a second dose of a composition comprising 2-10 mg/m² of SNDX-275 between day 8 and 29. In some embodiments, the SNDX-275 in said composition has a half-life of greater than about 24 hours.

Some embodiments provide a method of treating cancer in a patient, comprising administering a composition comprising 2-6 mg/m² of SNDX-275 to the patient. In some embodiments, said administration is oral.

Some embodiments provide a method of treating cancer in a patient, comprising administering to said patient a composition comprising SNDX-275 under such conditions and in sufficient amount to give rise to a Cₘₐₓ for SNDX-275 of from about 1 to about 5 ng/mL. In some embodiments, said administration is oral.

Some embodiments provide a method of treating cancer in a patient, comprising administering to a patient a composition comprising SNDX-275, wherein said composition produces a Cₘₐₓ of SNDX-275 in the patient of between 10 and 100 ng/mL. In some embodiments, the method comprises administering 6-10 mg/m² of SNDX-275 to the patient. In some embodiments, said administration is oral.

Some embodiments provide a method of treating cancer in a patient, comprising administering a composition comprising SNDX-275 to the patient, wherein said composition gives rise to an SNDX-275 AUC of about 80-210 ng·h/mL. In some embodiments, the administered composition contains 4-10 mg/m² of SNDX-275.

Some embodiments provide a method of treating cancer in a patient, comprising administering a first dose of a composition comprising 10-100 mg/kg of SNDX-275 on day 1 and administering a second dose of a composition comprising 10-100 mg/kg of SNDX-275 between day 8 and 29. In some embodiments, the SNDX-275 in said composition has a half-life of greater than about 24 hours.

Thus, some embodiments provide a method of treating cancer in a patient, comprising administering to the patient a first dose of SNDX-275, wherein the dose of SNDX-275 produces in the patient an area under the plasma concentration curve (AUC) for SNDX-275 in the range of about 100 to about 400 ng·h/mL. In some embodiments, a Cₘₐₓ of about 2.0 to about 50 ng/mL of SNDX-275 is achieved in the patient. In some embodiments, a Cₘₐₓ is obtained within 3-36 hours of administering the SNDX-275 to the patient. In some embodiments, the mean Cₘₐₓ across a patient population is in the range of about 4 to about 40 ng/mL. In some embodiments, the method further comprises administering a second dose of SNDX-275 to the patient. In some embodiments, the first dose is administered on day 1 and the second dose is administered on one of days 4-16. In some embodiments, the method further comprises administering a third dose of SNDX-275 to the patient. In some embodiments, the first dose is administered on day 1, the second dose on day 4-16 and the third dose on day 14-24. In some embodiments, the dose of SNDX-275 has a T_{1/2} of from about 20 to about 60 hours. In some embodiments, T_{1/2} for SNDX-275 is about 30 to about 50 hours. In some embodiments, the patient has a hematologic malignancy, a solid tumor or a lymphoma. In some embodiments, the patient has a hematologic malignancy. In some embodiments, the first dose of SNDX-275 contains no more than 7 mg/m² of SNDX-275. In some embodiments, the first dose of SNDX-275 contains no more than 6 mg/m² of SNDX-275. In some embodiments, the first dose of SNDX-275 contains from about 0.1 to about 6 mg/m² of SNDX-275. In some embodiments, the first dose is administered orally. In some embodiments, each dose is administered orally.

Some embodiments provide methods of treating cancer in a patient, comprising administering to the patient a flat dose of about 1 mg to about 10 mg of SNDX-275 no more than one time per week. In some embodiments, the flat dose is about 1 mg, 2 mg, 3 mg, 4 mg, 5 mg, 6 mg, 7 mg, 8 mg, 9 mg or 10 mg of SNDX-275, administered one time per week. In some embodiments, the flat dose is about 1 mg to about 6 mg of SNDX-275, administered no more than one time per week. In some embodiments, the flat dose is about 1 mg, 2 mg, 3 mg, 4 mg, 5 mg or 6 mg of SNDX-275, administered no more than one time per week. In some embodiments, the amount of SNDX-275 administered is sufficient to give rise to certain PK parameters in the patient. In some embodiments, the mean area under the plasma concentration curve of SNDX-275 is about 1 ng·h/mL to about 400 ng·h/mL. In some embodiments, the mean maximum plasma concentration of SNDX-275 is about 40 to about 60 ng/mL. In some embodiments, the mean time to maximum plasma concentration of SNDX-275 is about 0.5 to about 24 hours. In some embodiments, the SNDX-275 is administered orally. In some embodiments, the SNDX-275 is administered orally in the form of one or more tablets. In some embodiments, the SNDX-275 is administered orally in the form of 0.5, 1, 2, 3, 4, 5, 6, 7, 8, 9 or 10 mg tablets or a suitable combination of 2 or more thereof.

Some embodiments provide a method of treating cancer in a patient, comprising administering to the patient a flat dose of about 1 mg to about 10 mg of SNDX-275 no more than one time every other week. In some embodiments, the flat dose is about 1 mg, 2 mg, 3 mg, 4 mg, 5 mg, 6 mg, 7 mg, 8 mg, 9 mg or 10 mg of SNDX-275, administered one time every other week. In some embodiments, the flat dose is about 1 mg to about 6 mg of SNDX-275, administered one time every other week. In some embodiments, the flat dose is about 1 mg, 2 mg, 3 mg, 4 mg, 5 mg or 6 mg of SNDX-275, administered one time every other week. In some embodiments, the amount of SNDX-275 administered is sufficient to give rise to certain PK parameters in the patient. In some embodiments, the mean area under the plasma concentration curve of SNDX-275 is about 1 ng·h/mL to about 400 ng·h/mL. In some embodiments, the mean maximum plasma concentration of SNDX-275 is about 40 to about 60 ng/mL. In some embodiments, the mean time to maximum plasma concentration of SNDX-275 is about 0.5 to about 24 hours. In some embodiments, the SNDX-275 is administered orally. In some embodiments, the SNDX-275 is administered orally in the form of one or more tablets. In some embodiments, the SNDX-275 is administered orally in the form of 0.5, 1, 2, 3, 4, 5, 6, 7, 8, 9 or 10 mg tablets or a suitable combination of 2 or more thereof.

In some embodiments, the administered SNDX-275 produces an area under the plasma concentration curve (AUC) in the patient of about 100 to about 800 ng·h/mL. In some embodiments, the Cₘₐₓ for SNDX-275 is about 1 to about 100 ng/mL. In some embodiments, Tₘₐₓ is achieved from 0.5 to 24 hours after administration of SNDX-275.

When the HDAC inhibitor is co-administered with one or more additional compounds, the one or more additional compounds can be administered in a variety of cycles: the compound can be administered continuously, daily, every other day, every third day, once a week, twice a week, three times a week, bi-weekly, or monthly, while the second chemotherapeutic agent is administered continuously, daily, one day a week, two days a week, three days a week, four days a week, five days a week, six days a week, bi-weekly, or monthly. The compound and the second chemotherapeutic compound or cancer can be administered in, but are not limited to, any combination of the aforementioned cycles. In one non-limiting example, the compound is administered three times a week for the first two weeks followed by no administration for four weeks, and the second chemotherapeutic compound is administered continuously over the same six week period. In yet another non-limiting example, the compound is administered once a week for six weeks, and the second chemotherapeutic compound is administered every other day over the same six week period. In yet another non-limiting example, the compound is administered the first two days of a week, and the second chemotherapeutic compound is administered continuously for all seven days of the same week. The compound can be administered before, with or after the second chemotherapeutic compound is administered.

In addition to the administration of the compounds in cycles, the cycles themselves may consist of varying schedules. In some embodiments, a cycle is administered weekly. In other embodiments, a cycle is administered with one, two, three, four, five, six, or seven days off before repeating the cycle. In additional embodiments, a cycle is administered for one week with one, two, three, four, six, or eight weeks off before repeating the cycle. In further embodiments, a cycle is administered for two weeks with one, two, three, four, six, or eight weeks off before repeating the cycle. In still further embodiments, the cycle is administered for three, four, five, or six weeks, with one, two, three, four, six, or eight weeks off before repeating the cycle.

When a compound is administered with an additional treatment such as radiotherapy, the radiotherapy can be administered at 1 day, 2 days, 3 days, 4 days, 5 days, 6 days, 7 days, 14 days, 21 days, or 28 days after administration of at least one cycle of a compound. Alternatively, the radiotherapy can be administered at 1 day, 2 days, 3 days, 4 days, 5 days, 6 days, 7 days, 14 days, 21 days, or 28 days before administration of at least one cycle of a compound. In additional embodiments, the radiotherapy can be administered in any variation of timing with any variation of the aforementioned cycles for a compound. Additional schedules for co-administration of radiotherapy with cycles of a compound will be known in the art, can be further determined by appropriate testing, clinical trials, or can be determined by qualified medical professionals.

When a compound is administered with an additional treatment such as surgery, the compound is administered 1, 2, 3, 4, 5, 6, 7, 14, 21, or 28 days prior to surgery. In additional embodiments, at least one cycle of the compound is administered 1, 2, 3, 4, 5, 6, 7, 14, 21, or 28 days after surgery. Additional variations of administering compound cycles in anticipation of surgery, or after the occurrence of surgery, will be known in the art, can be further determined by appropriate testing and/or clinical trials, or can be determined by assessment of qualified medical professionals.

In addition to the aforementioned examples and embodiments of dosages, cycles, and schedules of cycles, numerous permutations of the aforementioned dosages, cycles, and schedules of cycles for the co-administration of a compound with a second chemotherapeutic compound, radiotherapy, or surgery are contemplated herein and can be administered according to the patient, type of cancer, and/or appropriate treatment schedule as determined by qualified medical professionals.

In various embodiments, a therapeutically equivalent amount of an HDAC inhibitor dose described herein is used.

mTOR Inhibitor Doses

In some embodiments, the amount of the mTOR inhibitor administered is a therapeutically effective amount. In various embodiments, there is synergy between the mTOR inhibitor and the HDAC inhibitor which allows for a lower dose of the mTOR inhibitor to be administered. In some embodiments, the synergy between the mTOR inhibitor allows for a lower dose of the HDAC inhibitor to be dosed. In some embodiments, the synergy between the mTOR inhibitor and the HDAC inhibitor allows for a lower dose of both the mTOR and the HDAC inhibitor to be dosed. In some embodiments, the synergy between the mTOR inhibitor and the HDAC inhibitor allows for the mTOR inhibitor to be dosed less frequently. In some embodiments, the synergy between the mTOR inhibitor and the HDAC inhibitor allows for the HDAC inhibitor to be dosed less frequently. In some embodiments, the synergy between the mTOR inhibitor and the HDAC inhibitor allows both the mTOR inhibitor and the HDAC inhibitor to be dosed less frequently.

In some embodiments, a therapeutically effective amount of the mTOR inhibitor is administered to the patient. In some embodiments, the administration may be repeated, e.g. on a twice daily schedule, a daily schedule, an every other day schedule, a every three day schedule, a every four day schedule, a weekly schedule, a biweekly schedule, a monthly schedule, etc. In some embodiments, the mTOR inhibitor is administered on one of the above mentioned schedules for 1, 2, 3, 4, 5, 6 or more weeks. In some embodiments, this round of dosing is then followed by a period in which no mTOR inhibitor is administered (wash-out period), which may be 1, 2, 3, 4 or more weeks. In some embodiments, the wash-out period is from about 1 day to about 3 weeks, or about 3 days to about 1 week, or about 1 week to about 2 weeks, or about 2 weeks to about 3 weeks. In some embodiments, the mTOR inhibitor is administered twice weekly for 4 weeks, followed by a 1, 2 or 3 week wash-out period. In some embodiments, the mTOR inhibitor is administered every 2, 3, or 4 days for 4 weeks, followed by a 1, 2 or 3 week wash-out period. In some embodiments, the mTOR inhibitor is administered once a week for 4 weeks followed by a 1, 2 or 3 week wash-out period. In some embodiments, the mTOR inhibitor is administered twice weekly for 6 weeks, followed by a 1, 2 or 3 week wash-out period. In some embodiments, the mTOR inhibitor is administered every 2, 3, or 4 days for 6 weeks, followed by a 1, 2 or 3 week wash-out period. In some embodiments, the mTOR inhibitor is administered once a week for 6 weeks followed by a 1, 2 or 3 week wash-out period. In some embodiments, the mTOR inhibitor is administered twice weekly for 2 weeks, followed by a 1, 2 or 3 week wash-out period. In some embodiments, the mTOR inhibitor is administered every 2, 3, or 4 days for 2 weeks followed by a 1, 2 or 3 week wash-out period. In some embodiments, the mTOR inhibitor is administered once a week for 2 weeks followed by a 1, 2 or 3 week wash-out period.

In some embodiments, flat dosing of the mTOR inhibitor may be employed. Suitable flat doses contemplated herein are about 0.5, 1, 2, 3, 4, 5, 6, 7, 8, 9, 10, 15, 20, 25, 30, 35, 40, or 50 mg of the mTOR inhibitor per dose. Such doses may be administered on one of dosing schedules described herein. In some embodiments, a dose of about 0.5, 1, 2, 3, 4, 5, 6, 7, 8, 9, 10, 15, 20, 25, 30, 35, 40, or 50 mg of the mTOR inhibitor is administered on a daily, every other day, twice-weekly, weekly (once per week) or biweekly (once every other week) dosing schedule, optionally with a rest period built in after a certain number of dosing cycles.

In some embodiments, the total weekly dosage range is about 5 mg to about 250 mgs. In various embodiments, the total weekly dosage range is about 5 mg to about 30 mg, or about 5 mg to about 25 mg, or about 5 mg to about 15 mg. In some embodiments, the total weekly dosage range is about 5-75 mgs. In some embodiments, the weekly dosage range is about 10 to about 70 mgs.

In certain embodiments, the therapeutically effective amount of the mTOR inhibitor is about 1-20 mgs. In some embodiments, the therapeutically effective amount of the mTOR inhibitor is about 1-15 mgs. In some embodiments, the therapeutically effective amount of the mTOR inhibitor is about 1-10 mgs.

In certain embodiments, the therapeutically effective amount of the mTOR inhibitor is about 0.01 to about 1,000 mg/m². In some embodiments, the therapeutically effective amount of the mTOR inhibitor is from about 0.1 to about 500 mg/m². In certain embodiments, the therapeutically effective amount of the mTOR inhibitor is about 0.2 to about 100 mg/m². In some embodiments, the therapeutically effective amount of the mTOR inhibitor is about 0.2 to about 20 mg/m². In some embodiments, the total weekly dosage range is about 1 mg to about 50 mg/m² per biweek.

In some embodiments, suitable dosages of the mTOR inhibitor are between about 0.1 to about 20 mg/m². In some embodiments, the suitable dosages of the mTOR inhibitor are between about 0.25 to about 10 mg/m², or about 0.25 to about 7 mg/m², or about 0.5 to about 5 mg/m².

In some embodiments, suitable dosages of an mTOR inhibitor are given twice daily during a 4 week treatment course. In some embodiments, suitable dosages of an mTOR inhibitor are given weekly during a 4 week treatment course for up to 6 courses in the absence of disease progression or unacceptable toxicity. In some embodiments, suitable dosages of an mTOR inhibitor are given once every 2 weeks during a 4 week treatment course. In some embodiments, suitable dosages of an mTOR inhibitor are given daily during a 4 week treatment course. Treatment cycles described herein can be monthly, weekly, or bi-weekly. Treatment cycles can be from one to twelve continuous monthly cycles or a patient may begin one cycle, cease treatment, and then undergo another cycle.

In some embodiments, suitable dosages of an mTOR inhibitor are given once daily for 5 days every 2 weeks. In some embodiments, the dosages range from 0.75 mg/m² per day to 24 mg/m² day.

In some embodiments, dosages of mTOR inhibitor are given daily in amounts ranging 0.5 mg to 10 mg.

In certain embodiments wherein the mTOR inhibitor is rapamycin, the therapeutically effective amount is about 0.5 to about 10 mg/day. In certain embodiments wherein the mTOR inhibitor is sirolimus, the therapeutically effective amount is about 0.5 to about 10 mg/day. In certain embodiments wherein the mTOR inhibitor is everolimus, the therapeutically effective amount is about 0.5 to about 10 mg/day. In certain embodiments wherein the mTOR inhibitor is temsirolimus, the therapeutically effective amount is about 0.5 to about 10 mg/day.

In certain embodiments wherein the mTOR inhibitor is rapamycin, the therapeutically effective dose is about 1 mg to about 10 mg. In some embodiments, the therapeutically effective dose is about 1 to about 6 mgs. In some embodiments, the therapeutically effective dose is about 1 mg, 2 mg, 4 mg, or 6 mg. In some embodiments, suitable dosages of rapamycin are given during a 13 day cycle. In some embodiments, a suitable dose of rapamycin is given once daily.

In certain embodiments, wherein the mTOR inhibitor is temsirolimus, a suitable dose of temsirolimus is given daily. In some embodiments, the suitable dose of temsirolimus is between about 1 mg to about 15 mgs delivered. In some embodiments, a dose of 1-15 mgs is delivered daily, every other day or every three days for at least two doses. In some embodiments, the suitable dose of temsirolimus is between about 1 mg and about 10 mgs. In some embodiments, a dose of 1-10 mgs is delivered daily, every other day or every three days for at least two doses. In some embodiments, the suitable dose of temsirolimus is between about 1 mg and about 7.5 mgs. In some embodiments, a dose of 1-7.5 mgs is delivered daily, every other day or every three days for at least two doses. In some embodiments, the suitable dose of temsirolimus is between about 1 mg and about 5 mgs. In some embodiments, a dose of 1-5 mgs is delivered daily, every other day or every three days for at least two doses. In some embodiments, a suitable dose of temsirolimus is given weekly.

In certain embodiments, wherein the mTOR inhibitor is temsirolimus, a suitable dose of temsirolimus is between about 10 mgs to about 250 mgs. In some embodiments, a dose of 10-250 mgs is administered weekly or every other week for at least two doses. In some embodiments, the suitable dose of temsirolimus is between about 10 mgs to about 100 mgs. In some embodiments, a dose of 10-100 mgs is administered weekly or every other week for at least two doses. In some embodiments, a suitable dose of temsirolimus is between about 10 mgs to about 70 mgs. In some embodiments, a dose of 10-70 mgs is administered weekly or every other week for at least two doses. In some embodiments, a suitable dose of temsiroliums is between about 10 mgs to about 50 mgs. In some embodiments, a dose of 10-50 mgs is administered weekly or every other week for at least two doses. In some embodiments, a suitable dose of temsiroliums is between about 10 mgs to about 30 mgs. In some embodiments, a dose of 10-30 mgs is administered weekly or every other week for at least two doses. In some embodiments, a suitable dose of temsirolimus is between about 10 mgs and about 20 mgs. In some embodiments, a dose of 10-20 mgs is administered weekly or every other week for at least two doses. In some embodiments, the treatment course is 4 weeks. In some embodiments, the treatment course is 3 weeks. In some embodiment, the treatment course if 2 weeks.

In certain embodiments, wherein the mTOR inhibitor is temsirolimus, a suitable dose of temsirolimus is 25, 75, or 250 mgs weekly. In certain embodiments, wherein the mTOR inhibitor is temsirolimus, a suitable dose of temsirolimus is 5 mgs or 10 mgs daily. In some embodiments, the suitable dose of temsirolimus is administered daily for at least two days.

In various embodiments, a therapeutically equivalent amount of an mTOR inhibitor dose described herein is used.

### Dosage Forms

The pharmaceutical composition may, for example, be in a form suitable for oral administration as a tablet, capsule, cachet, pill, lozenge, powder or granule, sustained release formulations, solution, liquid, suspension, for parenteral injection as a sterile solution, suspension or emulsion, for topical administration as an ointment, cream, lotions, sprays, foams, gel or paste, or for rectal or vaginal administration as a suppository or pessary. The pharmaceutical composition may be in unit dosage forms suitable for single administration of precise dosages. The pharmaceutical composition will include a conventional pharmaceutical carrier or excipient and the compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch or other cellulosic material, alginic acid and certain complex silicates and with binding agents such as sucrose, gelatin and acacia. Additionally, lubricating agents such as magnesium stearate, sodium lauryl sulfate and talc are often useful for tableting purposes. Other reagents such as an inhibitor, surfactant or solubilizer, plasticizer, stabilizer, viscosity increasing agent, or film forming agent may also be added. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Ester, Pa., 18th Edition (1990).

### Combination Therapies

The HDAC inhibitor/mTOR inhibitor combination therapies described herein may also be administered with another cancer therapy or therapies. As described above, these additional cancer therapies can be, for example, surgery, radiation therapy, administration of chemotherapeutic agents and combinations of any two or all of these methods. Combination treatments may occur sequentially or concurrently and the combination therapies may be neoadjuvant therapies or adjuvant therapies.

In some embodiments, the combinations described herein can be administered with an additional therapeutic agent. In these embodiments, the compound described herein can be in a fixed combination with the additional therapeutic agent or a non-fixed combination with the additional therapeutic agent.

In applications with administration of a therapeutic agent for treatment of side effects with the combination treatments as described, the therapeutic agent for treatment of side effects may be administered concurrently (e.g., simultaneously, essentially simultaneously or within the same treatment protocol) or sequentially, depending upon the nature and onset of the side effect, the condition of the patient, and the actual choice of chemotherapeutic agent and/or radiation to be administered in conjunction (i.e., within a single treatment protocol) with the compound/composition. For a non-limiting example, an anti-nausea drug may be prophylactically administered prior to combination treatment with the compound and radiation therapy. For another non-limiting example, an agent for rescuing immuno-suppressive side effects is administered to the patient subsequent to the combination treatment of compound and another chemotherapeutic agent. The routes of administration for the therapeutic agent for side effects can also differ than the administration of the combination treatment. The determination of the mode of administration for treatment of side effects and the advisability of administration, where possible, in the same pharmaceutical composition, is within the knowledge of the skilled clinician with the teachings described herein. The initial administration can be made according to established protocols known in the art, and then, based upon the observed effects, the dosage, modes of administration and times of administration can be modified by the skilled clinician. The particular choice of therapeutic agent for treatment of side effects will depend upon the diagnosis of the attending physicians and their judgment of the condition of the patient and the appropriate treatment protocol.

In some embodiments, therapeutic agents specific for treating side effects may by administered before the administration of the combination treatment described. In other embodiments, therapeutic agents specific for treating side effects may by administered simultaneously with the administration of the combination treatment described. In another embodiments, therapeutic agents specific for treating side effects may by administered after the administration of the combination treatment described.

In some embodiments, therapeutic agents specific for treating side effects may include, but are not limited to, anti-emetic agents, immuno-restorative agents, antibiotic agents, anemia treatment agents, and analgesic agents for treatment of pain and inflammation.

Anti-emetic agents are a group of drugs effective for treatment of nausea and emesis (vomiting). Cancer therapies frequently cause urges to vomit and/or nausea. Many anti-emetic drugs target the 5-HT₃ seratonin receptor which is involved in transmitting signals for emesis sensations. These 5-HT₃ antagonists include, but are not limited to, dolasetron (Anzemet®), granisetron (Kytril®), ondansetron (Zofran®), palonosetron and tropisetron. Other anti-emetic agents include, but are not limited to, the dopamine receptor antagonists such as chlorpromazine, domperidone, droperidol, haloperidol, metaclopramide, promethazine, and prochlorperazine; antihistamines such as cyclizine, diphenhydramine, dimenhydrinate, meclizine, promethazine, and hydroxyzine; lorazepram, scopolamine, dexamethasone, Emetrol®, propofol, and trimethobenzamide. Administration of these anti-emetic agents in addition to the above described combination treatment will manage the potential nausea and emesis side effects caused by the combination treatment.

Immuno-restorative agents are a group of drugs that counter the immuno-suppressive effects of many cancer therapies. The therapies often cause myelosuppression, a substantial decrease in the production of leukocytes (white blood cells). The decreases subject the patient to a higher risk of infections. Neutropenia is a condition where the concentration of neutrophils, the major leukocyte, is severely depressed. Immuno-restorative agents are synthetic analogs of the hormone, granulocyte colony stimulating factor (G-CSF), and act by stimulating neutrophil production in the bone marrow. These include, but are not limited to, filgrastim (Neupogen®), PEG-filgrastim (Neulasta®) and lenograstim. Administration of these immuno-restorative agents in addition to the above described combination treatment will manage the potential myelosupression effects caused by the combination treatment.

Antibiotic agents are a group of drugs that have anti-bacterial, anti-fungal, and anti-parasite properties. Antibiotics inhibit growth or causes death of the infectious microorganisms by various mechanisms such as inhibiting cell wall production, preventing DNA replication, or deterring cell proliferation. Potentially lethal infections occur from the myelosupression side effects due to cancer therapies. The infections can lead to sepsis where fever, widespread inflammation, and organ dysfunction arise. Antibiotics manage and abolish infection and sepsis and include, but are not limited to, amikacin, gentamicin, kanamycin, neomycin, netilmicin, streptomycin, tobramycin, loracarbef, ertapenem, cilastatin, meropenem, cefadroxil, cefazolin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, teicoplanin, vancomycin, azithromycin, clarithromycin, dirithromycin, erthromycin, roxithromycin, troleandomycin, aztreonam, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, benzolamide, bumetanide, chlorthalidone, clopamide, dichlorphenamide, ethoxzolamide, indapamide, mafenide, mefruside, metolazone, probenecid, sulfanilamides, sulfamethoxazole, sulfasalazine, sumatriptan, xipamide, democlocycline, doxycycline, minocycline, oxytetracycline, tetracycline, chloramphenical, clindamycin, ethambutol, fosfomycin, fusidic acid, furazolidone, isoniazid, linezolid, metronidazole, mupirocin, nitrofurantoin, platesimycin, pyrazinamide, dalfopristin, rifampin, spectinomycin, and telithromycin. Administration of these antibiotic agents in addition to the above described combination treatment will manage the potential infection and sepsis side effects caused by the combination treatment.

Anemia treatment agents are compounds directed toward treatment of low red blood cell and platelet production. In addition to myelosuppression, many cancer therapies also cause anemias, deficiencies in concentrations and production of red blood cells and related factors. Anemia treatment agents are recombinant analogs of the glycoprotein, erythropoietin, and function to stimulate erythropoesis, the formation of red blood cells. Anemia treatment agents include, but are not limited to, recombinant erythropoietin (EPOGEN®, Dynopro®) and Darbepoetin alfa (Aranesp®). Administration of these anemia treatment agents in addition to the above described combination treatment will manage the potential anemia side effects caused by the combination treatment.

Pain and inflammation side effects arising from the described herein combination treatment may be treated with compounds selected from the group comprising: corticosteroids, non-steroidal anti-inflammatories, muscle relaxants and combinations thereof with other agents, anesthetics and combinations thereof with other agents, expectorants and combinations thereof with other agents, antidepressants, anticonvulsants and combinations thereof; antihypertensives, opioids, topical cannabinoids, and other agents, such as capsaicin.

For the treatment of pain and inflammation side effects, compounds according to the present invention may be administered with an agent selected from the group comprising: betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocalne, EMLA Cream (Eutectic Mixture of Local Anesthetics (lidocaine 2.5% and prilocalne 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab) nitric oxide synthase inhibitors, particularly inhibitors of inducible nitric oxide synthase; and other agents, such as capsaicin. Administration of these pain and inflammation analgesic agents in addition to the above described combination treatment will manage the potential pain and inflammation side effects caused by the combination treatment.

### Kits for Co-Administration

As discussed above, in some embodiments, the mTOR inhibitor (e.g., temsirolimus or everolimus) and HDAC inhibitor (e.g., SNDX-275) may or may not be administered in combination with one or more active pharmaceutical ingredients in the treatment cancer. In particular, the mTOR inhibitor and HDAC inhibitor may be co-administered with a compound that works synergistically with either the mTOR inhibitor and/or the HDAC inhibitor and/or treats one of the sequelae of cancer or of cancer treatment, such as nausea, emesis, alopecia, fatigue, anorexia, anhedonia, depression, immunosuppression, infection, etc.

In some embodiments, the invention provides a kit including an HDAC inhibitor (e.g., SNDX-275) in a dosage form, especially a dosage form for oral administration. In some embodiments, the kit further includes an mTOR inhibitor (e.g., temsirolimus or everolimus) in a dosage form, especially a dosage form for oral administration. In specific embodiments, the HDAC inhibitor and the mTOR inhibitor are in separate dosage forms. In some embodiments of the invention, the kit includes one or more doses of an HDAC inhibitor (e.g., SNDX-275) in tablets for oral administration. In other embodiments, however, the dose or doses an HDAC inhibitor (e.g., SNDX-275) may be present in a variety of dosage forms, such as capsules, caplets, gel caps, powders for suspension, etc. In some embodiments of the invention, the kit includes one or more doses of an mTOR inhibitor (e.g., temsirolimus or everolimus) in tablets for oral administration. In other embodiments, however, the dose or doses of an mTOR inhibitor (e.g., temsirolimus or everolimus) may be present in a variety of dosage forms, such as capsules, caplets, gel caps, powders for suspension, etc.

In some embodiments, a kit according to the invention includes at least three dosage forms, one comprising an HDAC inhibitor (e.g., SNDX-275), one comprising an mTOR inhibitor (e.g., temsirolimus or everolimus) and the other comprising at least a third active pharmaceutical ingredient, other than the HDAC inhibitor and the mTOR inhibitor pharmaceutical ingredient. In some embodiments, the third active pharmaceutical ingredient is a second HDAC inhibitor. In other embodiments, the third active pharmaceutical ingredient is a second mTOR inhibitor. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. It is considered that the most convenient periods of time for which such kits are designed would be from 1 to 13 weeks, especially 1 week, 2 weeks, 1 month, 3 months, etc. In some specific embodiments, the each dose is physically separated into a compartment, in which each dose is segregated from the others.

In some embodiments, the kit according to the invention includes at least two dosage forms one comprising an HDAC inhibitor (e.g., SNDX-275) and one comprising an mTOR inhibitor (e.g., temsirolimus or everolimus). In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. In some specific embodiments, the each dose is physically separated into a compartment, in which each dose is segregated from the others.

In particular embodiments, the kit may advantageously be a blister pack. Blister packs are known in the art, and generally include a clear side having compartments (blisters or bubbles), which separately hold the various doses, and a backing, such as a paper, foil, paper-foil or other backing, which is easily removed so that each dose may be separately extracted from the blister pack without disturbing the other doses. In some embodiments, the kit may be a blister pack in which each dose of the HDAC inhibitor (e.g., SNDX-275), the mTOR inhibitor (e.g., temsirolimus or everolimus) and, optionally, a third active pharmaceutical ingredient are segregated from the other doses in separate blisters or bubbles. In some such embodiments, the blister pack may have perforations, which allow each daily dose to be separated from the others by tearing it away from the rest of the blister pack. The separate dosage forms may be contained within separate blisters. Segregation of the active pharmaceutical ingredients into separate blisters can be advantageous in that it prevents separate dosage forms (e.g. tablet and capsule) from contacting and damaging one another during shipping and handling. Additionally, the separate dosage forms can be accessed and/or labeled for administration to the patient at different times.

In some embodiments, the kit may be a blister pack in which each separate dose the HDAC inhibitor (e.g., SNDX-275), the mTOR inhibitor (e.g., temsirolimus or everolimus) and, optionally, a third active pharmaceutical ingredient is segregated from the other doses in separate blisters or bubbles. In some such embodiments, the blister pack may have perforations, which allow each daily dose to be separated from the others by tearing it away from the rest of the blister pack. The separate dosage forms may be contained within separate blisters.

In some embodiments, the third active pharmaceutical ingredient may be in the form of a liquid or a reconstitutable powder, which may be separately sealed (e.g. in a vial or ampoule) and then packaged along with a blister pack containing separate dosages of the HDAC inhibitor (e.g., SNDX-275) and the mTOR inhibitor (e.g., temsirolimus or everolimus). In some embodiments, the mTOR inhibitor (e.g., temsirolimus or everolimus) is in the form of a liquid or reconstitutable powder that is separately sealed (e.g., in a vial or ampoule) and then packaged along with a blister pack containing separate dosages of the HDAC inhibitor (e.g., SNDX-275). These embodiments would be especially useful in a clinical setting where prescribed doses of the HDAC inhibitor, mTOR inhibitor and, optionally, a third active pharmaceutically active agent would be used on a dosing schedule in which the HDAC inhibitor is administered on certain days, the mTOR inhibitor is administered on the same or different days and the third active pharmaceutical ingredient is administered on the same or different days from either or both of the HDAC and/or mTOR inhibitors within a weekly, biweekly, 2×weekly or other dosing schedule. Such a combination of blister pack containing an HDAC inhibitor, an mTOR inhibitor and an optional third active pharmaceutical agent could also include instructions for administering each of the HDAC inhibitor, an mTOR inhibitor and the optional third active pharmaceutical agent on a dosing schedule adapted to provide the synergistic or sequelae-treating effect of the HDAC inhibitor and/or the third active pharmaceutical agent.

In other embodiments, the kit may be a container having separate compartments with separate lids adapted to be opened on a particular schedule. For example, a kit may comprise a box (or similar container) having seven compartments, each for a separate day of the week, and each compartment marked to indicate which day of the week it corresponds to. In some specific embodiments, each compartment is further subdivided to permit segregation of one active pharmaceutical ingredient from another. As stated above, such segregation is advantageous in that it prevents damage to the dosage forms and permits dosing at different times and labeling to that effect. Such a container could also include instructions for administering an HDAC inhibitor, an mTOR inhibitor and the optional third active pharmaceutical ingredient on a dosing schedule adapted to provide the synergistic or sequelae-treating effect of the HDAC inhibitor and/or the third active pharmaceutical ingredient.

The kits may also include instructions teaching the use of the kit according to the various methods and approaches described herein. Such kits optionally include information, such as scientific literature references, package insert materials, clinical trial results, and/or summaries of these and the like, which indicate or establish the activities and/or advantages of the composition, and/or which describe dosing, administration, side effects, drug interactions, disease state for which the composition is to be administered, or other information useful to the health care provider. Such information may be based on the results of various studies, for example, studies using experimental animals involving in vivo models and studies based on human clinical trials. In various embodiments, the kits described herein can be provided, marketed and/or promoted to health providers, including physicians, nurses, pharmacists, formulary officials, and the like. Kits may, in some embodiments, be marketed directly to the consumer. In certain embodiments, the packaging material further comprises a container for housing the composition and optionally a label affixed to the container. The kit optionally comprises additional components, such as but not limited to syringes for administration of the composition.

In some embodiments, the kit comprises an HDAC inhibitor that is visibly different from the mTOR inhibitor. In certain embodiments, each of the HDAC inhibitor (e.g., SNDX-275) dosage form and the mTOR inhibitor (temsirolimus or everolimus) dosage form are visibly different from a third pharmaceutical agent dosage form. The visible differences may be for example shape, size, color, state (e.g. liquid/solid), physical markings (e.g. letters, numbers) and the like. In certain embodiments, the kit comprises an HDAC inhibitor (e.g., SNDX-275) dosage form that is a first color, an mTOR inhibitor dosage (e.g., temsirolimus or everolimus) form that is a second color, and an optional third pharmaceutical composition that is a third color. In embodiments wherein the first, second and third colors are different, the different colors of the first, second and third pharmaceutical compositions is used, e.g., to distinguish between the first, second and third pharmaceutical compositions.

In some embodiments, wherein the packaging material further comprises a container for housing the pharmaceutical composition, the kit comprises an HDAC inhibitor (e.g., SNDX-275) composition that is in a different physical location within the kit from an mTOR inhibitor composition. In further embodiments, the kit comprises a third pharmaceutical agent that is in a separate physical location from either the mTOR inhibitor composition or the HDAC inhibitor composition. In some embodiments, the different physical locations of HDAC inhibitor composition and the mTOR inhibitor composition comprise separately sealed individual compartments. In certain embodiments, the kit comprises an HDAC inhibitor composition that is in a first separately sealed individual compartment and an mTOR inhibitor composition that is in a second separately sealed individual compartment. In embodiments wherein the HDAC inhibitor composition and mTOR inhibitor composition compartments are separate, the different locations are used, e.g., to distinguish between the HDAC inhibitor composition and mTOR inhibitor compositions. In further embodiments, a third pharmaceutical composition is in a third physical location within the kit.

### Pharmacokinetics of SNDX-275

In various embodiments, the HDAC inhibitor (e.g., SNDX-275) is dosed in so as to minimize toxicity to the patient. In some embodiments, the HDAC inhibitor (e.g., SNDX-275) is dosed in a manner adapted to provide particular pharmacokinetic (PK) parameters in a human patient. In some embodiments, the HDAC inhibitor (e.g., SNDX-275) is dosed in a manner adapted to provide a particular maximum blood concentration (Cₘₐₓ) of the HDAC inhibitor (e.g., SNDX-275). In some embodiments, the HDAC inhibitor (e.g., SNDX-275) is dosed in a manner adapted to provide a particular time (Tₘₐₓ) at which a maximum blood concentration of the HDAC inhibitor (e.g., SNDX-275) is obtained. In some embodiments, the HDAC inhibitor (e.g., SNDX-275) is dosed in a manner adapted to provide a particular area under the blood plasma concentration curve (AUC) for the HDAC inhibitor (e.g., SNDX-275). In some embodiments, the HDAC inhibitor (e.g., SNDX-275) is dosed in a manner to provide a particular clearance rate (CL/F) or a particular half-life (T_{1/2}) for the HDAC inhibitor (e.g., SNDX-275). Unless otherwise specified herein, the PK parameters recited herein, including in the appended claims, refer to mean PK values for a cohort of at least 3 patients under the same dosing schedule. Thus, unless otherwise specified: AUC=mean AUC for a cohort of at least 3 patients; Cₘₐₓ=mean Cₘₐₓ for a cohort of at least 3 patients; Tₘ=mean Tₘₐₓ for a cohort of at least 3 patients; T_{1/2}=mean T_{1/2} for a cohort of at least 3 patients; and CL/F=mean CL/F for a cohort of at least 3 patients. In some embodiments, the mean is a cohort of at least 6 patients, or at least 12 patients or at least 24 patients or at least 36 patients. Where other than mean PK values are intended, it will be indicated that the value pertains to individuals only. Also, unless otherwise indicated herein, AUC refers to the mean AUC for the cohort of at least 3 patients, extrapolated to infinity following a standard clearance model. If AUC for a time certain is intended, the start (x) and end (y) times will be indicated by suffix appellation to “AUC” (e.g. AUC_{x, y}).

In some embodiments, the HDAC inhibitor (e.g., SNDX-275) is dosed in a manner adapted to provide maximum blood concentration (Cₘₐₓ) of the HDAC inhibitor (e.g., SNDX-275) of about 1 to about 135 ng/mL, especially about 1 to about 55 ng/mL, particularly about 1 to about 40 ng/mL of SNDX-275. In some embodiments, SNDX-275 is dosed in a manner adapted to provide maximum blood concentration (Cₘₐₓ) of SNDX-275 of about 1 to about 20 ng/mL, especially about 1 to about 10 ng/mL, particularly about 1 to about 5 ng/mL of SNDX-275. In some embodiments, SNDX-275 is dosed in a manner adapted to provide a Cₘₐₓ of 10-100 ng/mL. In various embodiments, the SNDX-275 is dosed in a manner adapted to provide a C of 10-75 ng/mL, or 10-50 ng/mL, or 10-25 ng/mL. In some embodiments, the SNDX-275 is dosed in a manner adapted to provide a Cₘₐₓ of less than about 50 ng/mL, or less than about 30 ng/mL, or less than about 20 ng/mL, or less than about 10 ng/mL, or less than about 5 ng/mL.

In some embodiments, the HDAC inhibitor (e.g., SNDX-275) is dosed in a manner adapted to provide a particular time (Tₘₐₓ) of about 0.5 to about 24 h, especially about 1 to about 12 hours. In some embodiments, the Tₘₐₓ is greater than about 24 hours. In some embodiments, the Tₘₐₓ is less than about 6 hours. In some embodiments, the Tₘₐₓ is between about 30 minutes and about 24 hours. In various embodiments, the Tₘₐₓ is between about 30 minutes and about 6 hours. In some embodiments, the Tₘₐₓ is

In some embodiments, the HDAC inhibitor (e.g., SNDX-275) is dosed in a manner adapted to provide a particular area under the blood plasma concentration curve (AUC) of the HDAC inhibitor (e.g., SNDX-275) of about 100 to about 700 ng·h/mL. In some embodiments, SNDX-275 is dosed biweekly under conditions adapted to provide an AUC of about 190 to about 700 ng·h/mL of SNDX-275. In some embodiments, SNDX-275 is dosed weekly under conditions adapted to provide an AUC of about 200 to about 350 ng·h/mL. In some embodiments, SNDX-275 is dosed biweekly under conditions adapted to provide an AUC of about 100 to about 500 ng·h/mL. In some embodiments, SNDX-275 is dosed under conditions adapted to provide an AUC of about 75-225 ng·h/mL.

In some embodiments, the terminal half-life (T_{1/2}) of the HDAC inhibitor (e.g., SNDX-275) is at least 48 hours. In some embodiments, the T_{1/2} is between about 48 hours and about 168 hours. In some embodiments, the T_{1/2} is between about 48 and 120 hours. In some embodiments, the T_{1/2} is between about 72 and 120 hours. In some embodiments, the T_{1/2} is between 24 and 48 hours.

### EXAMPLES

The following non-limiting, illustrative examples provide further elucidation of the embodiments disclosed herein.

### Example 1

### Human Clinical Trial of the Safety and Efficacy of Combination of HDAC Inhibitor and mTOR Inhibitor

Objective: To compare the safety and pharmacokinetics of administered HDAC inhibitor and mTOR Inhibitor.

Study Design: This will be a Phase I, single-center, open-label, randomized dose escalation study followed by a Phase II study in cancer patients with disease that can be biopsied (e.g., breast cancer, non-small cell lung cancer, prostate cancer, pancreatic cancer, colorectal cancer, head and neck cancer). Patients should not have had exposure to the HDAC inhibitor or the mTOR inhibitor prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive an mTOR inhibitor and HDAC inhibitor according to a pre-determined dosing regimen. Cohorts of 3-6 patients receive escalating doses of the mTOR inhibitor and the HDAC inhibitor until the maximum tolerated dose (MTD) for the combination of the mTOR inhibitor and the HDAC inhibitor is determined. Test dose ranges are initially determined via the established individual dose ranges for MS-275 and temsirolimus. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive the mTOR inhibitor as in phase I at the MTD determined in phase I and the HDAC inhibitor as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of the HDAC inhibitor and/or the mTOR inhibitor. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH. After completion of study treatment, patients are followed periodically for 4 weeks.

### Example 2

### Administration of SNDX-275 and Temsirolimus for Non-Small Cell Lung Cancer

According to Example 1, a Human Clinical Trial of the Safety and/or Efficacy of MS-275/temsirolimus combination therapy is performed. The cancer patients are rectum cancer patients who have not had exposure to either MS-275 or temsirolimus prior to the study entry and have not received treatment for their cancer within 2 weeks of beginning the trial. In conclusion, administration of a combination of MS-275 and temsirolimus will be safe and well tolerated by cancer patients. The combination of MS-275 and temsirolimus provides large clinical utility to these cancer patients.

### Example 3

### Administration of MS-275 and Temsirolimus for Treatment of Rectum Cancer

According to Example 1, a Human Clinical Trial of the Safety and/or Efficacy of MS-275/temsirolimus combination therapy is performed. The cancer patients have rectum cancer and have not had exposure to either MS-275 or temsirolimus prior to the study entry and have not received treatment for their cancer within 2 weeks of beginning the trial. In conclusion, administration of a combination of MS-275 and temsirolimus will be safe and well tolerated by cancer patients. The combination of MS-275 and temsirolimus provides large clinical utility to these cancer patients.

### Example 4

### Administration of MS-275 and Temsirolimus for Treatment of Solid Tumor Malignancies and Refractory Lymphoma

According to Example 1, a Human Clinical Trial of the Safety and/or Efficacy of MS-275/temsirolimus combination therapy is performed. The cancer patients have solid tumor malignancies or refractory lymphoma and have not had exposure to either MS-275 or temsirolimus prior to the study entry and have not received treatment for their cancer within 2 weeks of beginning the trial. In conclusion, administration of a combination of MS-275 and temsirolimus will be safe and well tolerated by cancer patients. The combination of MS-275 and temsirolimus provides large clinical utility to these cancer patients.

### Example 5

### Administration of MS-275 and RAD001 (Everolimus) for Treatment of Hepatocellular Carcinomas

According to Example 1, a Human Clinical Trial of the Safety and/or Efficacy of MS-275/RAD001 combination therapy is performed. The cancer patients have hepatocellular carcinomas and have not had exposure to either MS-275 or RAD001 prior to the study entry and have not received treatment for their cancer within 2 weeks of beginning the trial. In conclusion, administration of a combination of MS-275 and RAD001 will be safe and well tolerated by cancer patients. The combination of MS-275 and RAD001 provides large clinical utility to these cancer patients.

### Example 6

### Administration of MS-275 and Everolimus for Treatment of Metastatic or Unresctable Kidney Cancer

According to Example 1, a Human Clinical Trial of the Safety and/or Efficacy of MS-275/everolimus combination therapy is performed. The cancer patients have metastatic or unresectable kidney cancer and have not had exposure to either MS-275 or everolimus prior to the study entry and have not received treatment for their cancer within 2 weeks of beginning the trial. In conclusion, administration of a combination of MS-275 and everolimus will be safe and well tolerated by cancer patients. The combination of MS-275 and everolimus provides large clinical utility to these cancer patients.

### Example 7

### Administration of MS-275 and Temsirolimus for Treatment of Recurrent Malignant Glioma

According to Example 1, a Human Clinical Trial of the Safety and/or Efficacy of MS-275/temsirolimus combination therapy is performed. The cancer patients have recurrent malignant glioma and have not had exposure to either MS-275 or temsirolimus prior to the study entry and have not received treatment for their cancer within 2 weeks of beginning the trial. In conclusion, administration of a combination of MS-275 and temsirolimus will be safe and well tolerated by cancer patients. The combination of MS-275 and temsirolimus provides large clinical utility to these cancer patients.

### Example 8

### Administration of SNDX-275 and Temsirolimus for Renal Cell Cancer

According to Example 1, a Human Clinical Trial of the Safety and/or Efficacy of MS-275/temsirolimus combination therapy is performed. The cancer patients are renal cell cancer patients who have not had exposure to either MS-275 or temsirolimus prior to the study entry and have not received treatment for their cancer within 2 weeks of beginning the trial. In conclusion, administration of a combination of MS-275 and temsirolimus will be safe and well tolerated by cancer patients. The combination of MS-275 and temsirolimus provides large clinical utility to these cancer patients.

### Example 9

### Methods for Screening for mTOR Activity

One method of screening for mTOR inhibition is suppression of AKT signaling in acute myeloid leukemia (AML) cell lines.

Another method of screening for mTOR inhibition is collecting blood samples for secreted cytokine assays and enzyme-linked immunosorbent assay evaluation of surrogate tumor markers. Samples can be analyzed by phosphorylation assays of mTOR, AKT and S6Kinase, and by immunohistochemistry for expression of Ki67, PD1L (B7-H1), and PTEN.

Another method of screening for mTOR inhibition is assessing peripheral blood mononuclear cells isolated from blood samples and using a kinase assay to measure S6K activity.

Another method of screening for mTOR inhibition is collecting tumor tissue, normal skin, and oral mucosa and assessing by IHC staining of S6K and p-S6K, and by RT-PCR for cyclin D1 and p27.

Another method of screening for mTOR inhibition is selective inhibition of PTEN(−/−) cells.

### Example 10

### Parenteral Composition

An i.v. solution is prepared in a sterile isotonic solution of water for injection and sodium chloride (300 mOsm) at pH 11.2 with a buffer capacity of 0.006 mol/l/pH unit. The protocol for preparation of 100 ml of a 5 mg/ml an HDAC inhibitor and/or mTOR inhibitor for i.v. infusion is as follows: add 25 ml of NaOH (0.25 N) to 0.5 g of a first and/or second agent and stir until dissolved without heating. Add 25 ml of water for injection and 0.55 g of NaCl and stir until dissolved. Add 0.1N HCl slowly until the pH of the solution is 11.2. The volume is adjusted to 100 ml. The pH is checked and maintained between 11.0 and 11.2. The solution is subsequently sterilized by filtration through a cellulose acetate (0.22 μm) filter before administration.

### Example 11

### Oral Composition

A pharmaceutical composition for oral delivery is prepared by mixing 100 mg of an HDAC inhibitor and/or mTOR inhibitor with 750 mg of a starch. The mixture is incorporated into an oral dosage unit, such as a hard gelatin capsule or coated tablet, which is suitable for oral administration.

Many modifications, equivalents, and variations of the present invention are possible in light of the above teachings, therefore, it is to be understood that within the scope of the appended claims, the invention may be practiced other than as specifically described.

What is claimed is:

1. A method of treating cancer in a patient, comprising administering an HDAC inhibitor and an mTOR inhibitor.

2. The method of claim 1, wherein the HDAC inhibitor is a Class I HDAC inhibitor.

3. The method of claim 1, wherein the HDAC inhibitor is SNDX-275.

4. The method of claim 3, wherein the administration of SNDX-275 provides a mean area under the blood plasma concentration curve of SNDX-275 of about 25 to about 700 ng·h/mL

5. The method of claim 3, wherein the administration of SNDX-275 provides a mean area under the plasma concentration curve of SNDX-275 of about 100 ng·h/mL to about 400 ng·h/mL.

6. The method of claim 3, wherein the administration of SNDX-275 provides a mean area under the plasma concentration curve of SNDX-275 of about 150 ng·h/mL to about 350 ng·h/mL.

7. The method of claim 3, wherein the administration of SNDX-275 provides a mean area under the plasma concentration curve of SNDX-275 of about 75 to about 225 ng·h/mL.

8. The method of claim 3, wherein the mean maximum plasma concentration of SNDX-275 is between about 1 and about 50 ng/mL.

9. The method of claim 3, wherein the mean maximum plasma concentration of SNDX-275 is between about 5 and about 25 ng/mL.

10. The method of claim 3, wherein the mean ½ life of the SNDX-275 is greater than about 24 hours.

11. The method of claim 3, further comprising detecting a drug-related toxicity in the patient and subsequently administering to the patient a reduced dose of SNDX-275.

12. The method of claim 3, wherein the dose of SNDX-275 is about 1 mg to about 6 mg.

13. The method of claim 3, wherein the SNDX is administered once a week.

14. The method of claim 3, wherein the SNDX is administered once every two weeks.

15. The method of claim 3, wherein the mean time to maximum plasma concentration of SNDX-275 is about 0.5 to about 24 hours.

16. The method of claim 3, wherein the SNDX-275 is administered orally in the form of one or more tablets.

17. The method of claim 3, wherein the SNDX-275 is administered orally in the form of 0.5, 1, 2, 3, 4, 5, 6, 7, 8, 9 or 10 mg tablets or a suitable combination of two or more thereof.

18. The method of claim 3, wherein the mTOR inhibitor is selected from temsirolimus (CCI-779), everolimus (RAD001), deforolimus (AP23573), AP21967, biolimus, AP23102, zotarolimus (ABT 578), sirolimus (Rapamune), or tacrolimus (Prograf).

19. The method of claim 1, wherein the mTOR inhibitor is administered in an amount of about 0.5 to about 10 mg/day.

20. The method of claim 1, wherein the mTOR inhibitor is administered in an amount of about 0.9 to about 4 mg/day.

21. The method of claim 1, wherein the cancer is lung cancer, multiple myeloma, gynecologic malignancies, non-hodgkins lymphoma, Hodgkin's disease, leukemia, melanoma, breast cancer, prostate cancer, kidney cancer, head cancer, neck cancer, renal cell cancer, or a solid tumor.

22. A method of treating cancer in a patient, comprising:
(a) administering to the patient a first dose of 3-10 mgs of SNDX-275 and a second dose of 3-10 mgs of SNDX-275, wherein the second dose of SNDX-275 is administered within 1-3 weeks of the first dose of SNDX-275; and
(b) administering at least one dose of an mTOR inhibitor, wherein the mTOR inhibitor is administered within the three weeks of the first dose of SNDX-275.

23. The method of claim 22, wherein administration of the first dose of SNDX-275 provides a mean area under the blood plasma concentration curve of SNDX-275 of about 25 to about 700 ng·h/mL

24. The method of claim 22, wherein administration of the first dose of SNDX-275 provides a mean area under the plasma concentration curve of SNDX-275 of about 100 ng·h/mL to about 400 ng·h/mL.

25. The method of claim 22, wherein administration of the first dose of SNDX-275 provides a mean area under the plasma concentration curve of SNDX-275 of about 150 ng·h/mL to about 350 ng·h/mL.

26. The method of claim 22, wherein administration of the first dose of SNDX-275 provides a mean area under the plasma concentration curve of SNDX-275 of about 75 to about 225 ng·h/mL.

27. The method of claim 22, wherein the mean maximum plasma concentration of SNDX-275 is between about 1 and about 50 ng/mL.

28. The method of claim 22, wherein the mean maximum plasma concentration of SNDX-275 is between about 5 and about 25 ng/mL.

29. The method of claim 22, wherein the mean 12 life of the SNDX-275 is greater than about 24 hours.

30. The method of claim 22, further comprising detecting a drug-related toxicity in the patient and subsequently administering to the patient a reduced dose of SNDX-275.

31. The method of claim 22, wherein the SNDX is administered once a week.

32. The method of claim 22, wherein the SNDX is administered once every two weeks.

33. The method of claim 22, wherein the mean time to maximum plasma concentration of SNDX-275 is about 0.5 to about 24 hours.

34. The method of claim 22, wherein the SNDX-275 is administered orally in the form of one or more tablets.

35. The method of claim 22, wherein the mTOR inhibitor is selected from temsirolimus (CCI-779), everolimus (RAD001), deforolimus (AP23573), AP21967, biolimus, AP23102, zotarolimus (ABT 578), sirolimus (Rapamune), or tacrolimus (Prograf).

36. The method of claim 22, wherein the mTOR inhibitor is sirolimus.

37. The method of claim 36, wherein the sirolimus is administered in an amount of about 0.5 to about 10 mg/day.

38. The method of claim 36, wherein the sirolimus is administered in an amount of about 0.9 to about 4 mg/day.

39. The method of claim 22, wherein the cancer is lung cancer, multiple myeloma, gynecologic malignancies, non-hodgkins lymphoma, Hodgkin's disease, leukemia, melanoma, breast cancer, prostate cancer, kidney cancer, head cancer, neck cancer, renal cell cancer, or a solid tumor.

40. The method of claim 1, wherein the cancer is of epithelial origin.

41. The method of claim 1, wherein the cancer is a hematological cancer.

42. The method of claim 22, wherein the cancer is of epithelial origin.

43. The method of claim 22, wherein the cancer is a hematological cancer.
